SEQUENCE LISTING (10 sequences)

SEQ ID NO: 1 - DNA, 1754 bp, Arabidopsis thaliana
atttccagaagcggcaacgatggagacaacaatcggagattctatgacgatgatgatgacggagatgtgaccggtatgagctcaagtagacgaaatctccaaacgattcagagcttcaaagcttcagatctgtttccttggatcggttaaaaagagttttaaggttgatttttctttccacaccatttggcgactcgtacatcgaactcgctgatcggaaaatactcccggatagaaattgaaccgattagtttgatgagtgagtgacttgttacttcgatttgtagctgagtgtgtcactgctcaagtgtgtgaagaaggagaaaatgccgaggccaggaccaagaccttacgagtgtgtgaaacgagcttggcatagcgatcgtcatcaacctattcgtggttcgattattcgccagattttcaggtttttttcattcactcatctttctcaaaaccgtacgctagggtttctttgtatcttaatttggggttttctttgtttgatttcagactcgcaatggaagcacatagcgcagcgacgaggaagaacaaagaatggcaggagaaattgcctgtggttgtgttaaaagctgaagaaatcatgtactctaaagccaattccgaggttttaaacaaattttccttctctttttttagtgtgttttcagcttcacaaaatgacaattgggtttcggtttccattaggaagagtatacagatgctgatactatgtggaatagagtaaatgatgccattgatacaattattagaagagatgagagtacagaaactggtcctcttttgcctccatgtgttgaaggtaattgtcatttttgtttctctctttagctctagtgatccttattgaaactacttagaagttagtgctttgtactgtttattgtttgttgatgtggtttttttttatatggttagctgctcttaatttgggatgtattgctgtaagagcttcaagaagccaacgacatagtagcggaaggacttacctcggtcctaaaattcaggaaccggtttctgcgtcgactaatgagccgtcttaccatcatgaatatcgtcagcaagctcaacaatcatcaaccaaaccaagccagacagttcaagctgcagtccctgttgatgtacttgataacagcaacaagcgtgtagctactcctcgcggttacccgtttcttcatgaatccatgcagatgcatcaaaagccattggcaattagacaaggaactggtcctgcttctgctcctgctccagctccagtcaacttgggttcggtctatcctttgtattacgaaggtaataatcaaacgcagcaagctgacatgtcttttagagtccctgaagcgcccataatcattggcatgcctattggtataaagccctcagaagaagcaactgagagggtctgtgatttatccttgaggctcggtatatcttcagaaccgtctacaagaatagatgtcgggtctagtcgggcttacccgggaagaaaccaagaagagttatgtttattcgcagaggtaaagaagaatgataggtttgaatggttttcaaatagtgaaggtcagaattcagattcgagagttaaaaagcacagaactttgtgtggagacttcctttaggacttgcttagttgttgtaaaagcttcaggtttattggtttactattttattgactctactagtcttagcgttcgcttcaagttttataataaagatccatggattgatcttaa

SEQ ID NO: 2 - DNA, 1032 bp, Arabidopsis thaliana
atgccgaggccaggaccaagaccttacgagtgtgtgaaacgagcttggcatagcgatcgtcatcaacctattcgtggttcgattattcgccagattttcagactcgcaatggaagcacatagcgcagcgacgaggaagaacaaagaatggcaggagaaattgcctgtggttgtgttaaaagctgaagaaatcatgtactctaaagccaattccgaggaagagtatacagatgctgatactatgtggaatagagtaaatgatgccattgatacaattattagaagagatgagagtacagaaactggtcctcttttgcctccatgtgttgaagctgctcttaatttgggatgtattgctgtaagagcttcaagaagccaacgacatagtagcggaaggacttacctcggtcctaaaattcaggaaccggtttctgcgtcgactaatgagccgtcttaccatcatgaatatcgtcagcaagctcaacaatcatcaaccaaaccaagccagacagttcaagctgcagtccctgttgatgtacttgataacagcaacaagcgtgtagctactcctcgcggttacccgtttcttcatgaatccatgcagatgcatcaaaagccattggcaattagacaaggaactggtcctgcttctgctcctgctccagctccagtcaacttgggttcggtctatcctttgtattacgaaggtaataatcaaacgcagcaagctgacatgtcttttagagtccctgaagcgcccataatcattggcatgcctattggtataaagccctcagaagaagcaactgagagggtctgtgatttatccttgaggctcggtatatcttcagaaccgtctacaagaatagatgtcgggtctagtcgggcttacccgggaagaaaccaagaagagttatgtttattcgcagaggtaaagaagaatgataggtttgaatggttttcaaatagtgaaggtcagaattcagattcgagagttaaaaagcacagaactttgtgtggagacttcctttag

SEQ ID NO: 3 - DNA, 1370 bp, Arabidopsis thaliana
actttgtctttttttctctctctctctctctctcatagaaaaggaagtggtggaagatgccgaggccagggccaagaccgtacgactgtatcagaagagcttggcatagtgacagacaccaacccatgaggggtttgctcatccaagagattttcaggtcatcatcatcatctctctctcactttggatatctatctgtgtatctataggtttgtgtgtgtgcatgtctttctttagctcaattgttctttgaactatttgtaggattgtttgtgagattcacagccaatccaccaggaaaaacacagaatggcaagagaagctccctgttgttgtcttgagagctgaagaaatcatgtattccaaagccaattctgaggtttttcttccaacattttaattacaaaggattgtttctaaatagtatttcgattttcattttttgctttttattgccctctttgaacagtgtgactgatagtctctggtaatctgtactaaaccttggattttatggagttgatagagtggatttgtagctaatgtgttcatttggaatatgaatattttatatacttcttgtgggtttgatgttaggctgagtatatggacatgaagacccttttagaccgtacaaacgacgccatcaacacaatcatacgacttgatgagaccaccgaaacaggggaatttcttcagccttgtattgaaggtatacaatacaatctgtatattcgagctttcggggattattggctcagatcaatgagcaagtcttgtttattatgtagctgcattgcatttggggtgcacaccaaggagagcttcaaggagccaacggaacattaacccgagatgttatcttagccaagactcaactaacttggacaacatgttgtcccaagtattcatgaaaccaaacaactttgctccaaagaatcttgcagtagcacaagagaattgccctgtctccaagtactctgcctaccctttatgctattcattccggccaatcagtgactcgtgtaagtccaagaacagtagaccggcgagtctcattgacgcaacaaatggcattacctttggtgggtgtgaatgtgatctatctctgcgcttaggtcctcttggacctcctactcaaaaacgaagtaagataagcagcaacaataacaactgaaaaggagagcaagattgggttttgattagcaagtcttgtgttagaagaatgtgtgcttagttaggccttttagtctcctctctagaccctaggtttaggtttgtgaacatgtaaatagagagacgaagtaacaaaacaggagatgaagtcgttttcttctacctgcatcaatctcgaatcatcagacttcacg

SEQ ID NO: 4 - DNA, 717 bp, Arabidopsis thaliana
atgccgaggccagggccaagaccgtacgactgtatcagaagagcttggcatagtgacagacaccaacccatgaggggtttgctcatccaagagattttcaggattgtttgtgagattcacagccaatccaccaggaaaaacacagaatggcaagagaagctccctgttgttgtcttgagagctgaagaaatcatgtattccaaagccaattctgaggctgagtatatggacatgaagacccttttagaccgtacaaacgacgccatcaacacaatcatacgacttgatgagaccaccgaaacaggggaatttcttcagccttgtattgaagctgcattgcatttggggtgcacaccaaggagagcttcaaggagccaacggaacattaacccgagatgttatcttagccaagactcaactaacttggacaacatgttgtcccaagtattcatgaaaccaaacaactttgctccaaagaatcttgcagtagcacaagagaattgccctgtctccaagtactctgcctaccctttatgctattcattccggccaatcagtgactcgtgtaagtccaagaacagtagaccggcgagtctcattgacgcaacaaatggcattacctttggtgggtgtgaatgtgatctatctctgcgcttaggtcctcttggacctcctactcaaaaacgaagtaagataagcagcaacaataacaactga

SEQ ID NO: 5 - DNA, 1212 bp, Glycine max
atgccacgcccagggccaaggccttacgagtgcgtgagaagggcttggcacagcgagcgccaccaacccgtgagaggttccatcattcagcagattttcagggtcgtcaatgacgctcacagtcccgcgaccaagaagaacaaggaatggcaagagaagctccccgtggtggttctcaaggccgaagaaatcatgtactccaaagccaattccgaggcagagtacttgaatcctgatacgctctgggaccgcctcaatgatgccgttaacacaatcatacggagagatgagaccaccgagacaggagacctcttgcccccatgtgttgaagctgcacttaacctgggatgcaaacctgtgagaacttccagaagtgatcggcacaataacccaaggacttacctttcttcaaggattcaacaacctccctctgtgtctcataaacctgatgctgggaatcccttgaactatgccaaagtcacaactcctgctgtctcaccaattcctgtgccagattctacccaccaaaatagcaagctcatgggttcctctaactaccctttctcagaaggcctcccttgtcatcgccagccattgacaaaggaagctagaccctcattgaacttgggttcagtttacccattgtactatggttatgaagccagagaacctcaacctaggacgactgctagagataccacgtgttctgatacaatctttgttggcagaccagtcattccagtccctgagccttcaggaattggtctattggaaaacttctcctatggtagattccatcacgttgcaaacagaataggaaaagagattgctgttggcactcaggaggcagcaccagatagggaatgtgacttgtctttgaggttgggtcaatgtctgcatccgtgttcaagcagcaaaagtagttcagcgtatgaagtagatgatgttggtttaggagtttcaccgggaagcagcaggtttagccatttatccttgcagaaatatagggagttctgtttttatcctagagagacaggttatggcaccattgagtccacttctggtaaatgtaatgtagagggtgaagatcagaacttagaggcaactcttaggaagcgcaaagcacctttaggtaacaacgaagaagatgggcaattttgccggaatctaggggtcccatccaatcgttttactggtaggccaggttcgtag

SEQ ID NO: 6 - DNA, 1290 bp, Gossypium raimondii
atgccgagaccaggccctagaccttacgagtgtgtaagaagagcttggcatagcgaaagacaccaacccatgagaggttccatcattcagcagattttaaggctagctattgaaactcatagcacagctactaaaaagaataaggagtggcaagataaaattattactgtcatcgtcaaagccgaggagattatgtattccaaagccaattctgaggctgaatacaccaacccggaaaccttatgggaccgtgttaatgatgccatcaacacgattatccgtcgagatgagagcactgaaacaggagagcttttgccaccttgtgttgaagcggcccttaacctggggtgctatccagtgagagcttcaaggagccaacgacacagtaatcctaggacttaccttacgccaagagcacccgaaactgttcctgcaactcctaggattttggataacggtagtgaggataggtgtcctcagttatccccggttcagtctggtagtcagcttgcaagaatagcaaccaatgtgaattcgaacacttcggtttcacaagctaaccgtcataattatccgtttctacctcaaaattgcccatctggccatgaccaattgatgaggatggaaactaacagtccatcgaactcaggtcaagtttaccccctttattatggaattcactatcaaaatgcagagtcccaaacaggttctccagtgcaggaaaatctagtgtctgatacgataatagttgggagacctattggttcatccgtgttagagccagctgaaatgggttccttgcagaatttcttttcctcttccaatgttgatattggtggtaagcgaattgggctgcaagatatcagacacactgatgagaagttacttgggaaagaatgtgatttgtccttgcggttgggtctattttctgacccttgtatgcaagtagaaaagaagtcactttgtgaaaccacagatgttggtcccagcaacactcaagatgggggcaaacttagtgatgtttttcaacaaaagagtaaagagatccccttttttccagagaggactgttaatgaccattttgaatccttttcaagaaagtgctttatggaaaatgaaggtcataatgtaggggcagcaacgagaaagcgtaaggcaacatttggaggcaactcagaagatgaacaattttgcaagcaaccagggtcctcatctaataatagaagacatggaccagataagcatttgctatatcacttctcggagatcaggatggcaagaggaatctaa

SEQ ID NO: 7 - DNA, 1275 bp, Populus balsamifera subsp. trichocarpa
atgccaaggccagggccaaggccttatgagtgtgtgagaagagcttggcatagtgataggcacaaacccatccgaggttccatgattggacaaattttaaggatggcctatgacactcacagtgctgcaactaaagggaacagggagtggcaagataagcttcttcttgtggtttacaaagcagaggaaatcatgtactctaaagccaactctgaggctgagtatatgaaccaagacactttttgggacagagtaaatgatgccatcaacaccattattaggagagatgagagcactgaaactggagatcttttgcctccctgtattgaagctgcccttaatttgggatgcaaagtggagagagcttcgagaagtcaacgacacagcaatcccaggagctacctcagcccgagaacacaagaatcggcgtctgtacctcctagagctatagatagaactcatgatgaacaaggccttcagttaatgccagttcactctatcaatcaattgaatattgcaagagacacggcaactgggaatccagatctttcggtttcagagtctaatcatcatttggctgagaatagtaatgttgcttacagttaccctttcttgtatgagaatatcccacctggcagtaatcagctgacaacaagggaagccgacatgcaccagaactttggttcagtttatcccttgtattgtggaaatcaatatcaaatcgaagcgtctgatgtggtctcccaatttccgatgaagacgaaatccaacactatatttgtgggcaaacctattggtacatctgttgcaccacatagggaaatgggtgtcttgcagacagttttctcatgttccagtgctgaagttggttccacgagaatcacacaagcagatttcagaaatacccatgacaagccaactggcacacaatgtgatttatccttgaggctgggtctatactcagacccgggcatgagcatagaaagaaatcaagctcaagaaaatgaaaatgctggctcaagcagatttcaagaaagagacaagtttagtgttttttctcaacaaagaaacaaggaattctgtttttttcctggtacaagtactagggatccctctgggtcctgttcggttaagtgggtttcagagggtgatgatcagaatttggagacaaccatcagaaagcgcaaagcgcccttcagagacaatgcagaggatggacatttttgctggcagtccaacatgttcattggtcgaatagaagggccaggtttgtag

SEQ ID NO: 8 - DNA, 828 bp, Glycine max
atgccccgcccaggtcccaaaccttacgattgcgtcaaaagagcttggcacagtgaaatacaccaacccatcagaggaactctcattcaagaaattttcagggttgtcaacgagatacatgggtcttctactaagaagaacaaagagtaccaagagaagctacccgttgttgtgctcagggcagaggaaattatttactctaaagccaattccgaggctgaatacatggaccttacaacacttttggaaagaacaaatgatgctattgatacaataattcgacgtgatgagcatacagaaactggagagtatttgcgcccttgcatcgaagctgctctaagtttgggttgctccttaacaaaagccacaaggagtcaacgcaacaaccaacgatgttacttaagtcgcagcactgaagaggttccaaaactttcttatggcacgttgcataataccgcaaatacgaaggatcataataacacaaggtctcaatgtgtgtccgagaatgtaccttcagctagtaaaaagcaatgcgtggaacaccaaccgccaccaaaattgttttcagtttatcctctgtactatgggaacaacaacaacatccaacttggcaattcacaacatcatcatgggttcaaagtatcacatgtatctgtttctcacactggtggacctgcccttgtgggtggtgatggtgctaggaatctactagcccataacttaaaaaattcttcaaatgggggatcccaatcattcattattaatggggattttgagaatccatgcacaagaaaatgtgatctatcattgaggttaggacccccttaa

SEQ ID NO: 9 - DNA, 1200 bp, Gossypium raimondii
atgcctagacctggtccaagaccttatgtctgtgagagaagagcttggcacagtgatagacaccaacccatgaggggttctctcattcaggaaattttcagagttgtaaatgagattcatagctcagcaacaaagaagaacaaggaatggcaagagaagctccctgatgttgtcttaaaagcagaagaaattatgtattccaaagccaattctgaggctgagtacatggacattaaaaccctctgggatcgaacaaatgatgccattaacacaatcattagacgtgatgagagtactgaaactggagagcttcttcaaccttgtattgaagctgctcttaatttgggctgcactgcgagaaggactttgcggagccaacggaattgtagtccaaggtcttatcttaacccgggtgctcaaaaagcagagggcactactctaggaaatctcataactaattcacattgcatggctagtgattccagcttcctaaaacacacaaccgtgaacatgacggatatgggctctgaagctcagaaacacattgctcagaacggtaaccgtggcactgacaagttttcttttgcatcaaacaatagtcctcttgctagtaatgtagagaagcatcctccaaatacctattcagtttatcctcttttctatggaaaccaccttaaagttgaagaacagcggcatggttatggaatctcaccaaagtcattttctaacacagtagagcctgccatgatgggtgtcattcatagtctcttctctcctgatgtggactcctcaaataagatgaaccaaacagatgttagaaatacttctaacaacccacatgaaattccctgtgacctatcactgcggctgggccctctctcaactccttgcctgagtgctggaaacagcaggcacaaggaaattaaaaacaccgattccacctttctagagtggaacaagttcagttatctgacacccccaattgatgaaagtttatcttccttacccaggagcaacagagatgcccccttgaacccgtattcaaacgaacggaatcttgaaggtggacatatggatgtagatgcaacattgagcaagcgaaagacaatttatggtccaccagtcgatcagcaattttgtttgtcaccaaagcttccttgcagcgaattaactggaagaatgaagcgtgtaggatcctag

SEQ ID NO: 10 - DNA, 1224 bp, Populus balsamifera subsp. trichocarpa
atgcctagaccaggaccaagaccttatgaatgtgttaggagagcttggcacagtgatagacaccaacccatcagaggttctctgattcaagaaattttccgacttgttaatgaggctcatagctcaacaactaagaagaacaaggaatggcaagagaagctcccagttgttgtcctgaaagcagaagaaattatgtactcgaaagccaattctgaggctgaatacatggagcttaagacactttgggacagaacaaatgatgccattaataccataattcggcgtgatgagagtacggaaattggagagcttcttcaaccttgcattgaagctgcccttaatttgggctgcacaccgagaagagcctcaaggagccaacggaattgcaatccgagcttttaccttagtcctagcactcaggaaccaaacactttgtcctctggttctgtgcacagtgccattcaggctaatcgtacatccaactcgcatgtcctacctaattattcaagcatggtaaaacccataatcatgaattccacccctccaggctcagaatcacaagactttgttggccaaagcaatggcacttctaacagatttctttttatagatgatagcattcctctatctaacgctaaccaatgtttacccttgggaaattaccgtataccaagcttgtgctcggtttaccctttatactatggttgttgcctagaaccacagcgaggttgtggagctcttcctaagacttttcctggcaccatggagcctgtcaaggtggctgttatgcaaaacttcttcccttgtaacgaggacactcctgttaaaacctgccatgctgatcacaaggacagtcctttgcaaccacaagaaattgggtgcgatctatcattgaggttgggttctctcccggctccaatgctgagtgtgaagaccaagcaactcaaagatgccaaagacggtggtcatgattgttctcaagaggggggtaaggttgatgattggatgccccaagtggacaaggagttacctttcttcactagggtcaatgtggctgacccgttagtttcacattcaagcaaatcgagggaacatgtaaatatagatgagacaaagaaaaaacgcaaggcggtattggatcaccatgttgaggatcaattttgctggcagccaaagcttcattgtaatcagttaacttgcagaatgaaaagtgccggttcgtag

(19) United States
(12) Patent Application Publication
(10) Pub. No.: US 2017/0159065 A1
(43) Pub. Date: Jun. 8, 2017

(54) MEANS AND METHODS TO INCREASE PLANT YIELD

(71) Applicants: VIB VZW, Gent (BE); Universiteit Gent, Gent (BE)

(72) Inventors: Dirk Gustaaf Inzé, Moorsel-Aalst (BE); Alain Goossens, Lokeren (BE); Nathalie Gonzalez, Merelbeke (BE); Laurens Pauwels, Gentbrugge (BE); Alexandra Baekelandt, Ronse (BE)

(21) Appl. No.: 15/321,636

(22) PCT Filed: Jul. 8, 2015
(86) PCT No.: PCT/EP2015/065607

§ 371 Date: Dec. 22, 2016

(30) Foreign Application Priority Data
Jul. 8, 2014 (EP) 14176109.8

(51) Int. Cl. C12N 15/82

(52) U.S. Cl. CPC C12N 15/8218; C12N 15/8261

(57) ABSTRACT

This disclosure relates to plants having a decreased expression of the KIX8 and KIX9 genes that result in an increased yield, particularly an increased leaf biomass. The disclosure provides plants and chimeric genes that can be used to decrease the combined KIX8/KIX9 gene expression.

### CROSS-REFERENCE TO RELATED APPLICATIONS

This application is a national phase entry under 35 U.S.C. §371 of International Patent Application PCT/EP2015/065607, filed Jul. 8, 2015, designating the United States of America and published in English as International Patent Publication WO 2016/005449 A1 on Jan. 14, 2016, which claims the benefit under Article 8 of the Patent Cooperation Treaty to European Patent Application Serial No. 14176109.8, filed Jul. 8, 2014.

### TECHNICAL FIELD

This application relates to the field of agricultural biology. In particular, this disclosure relates to novel plants that have a reduced expression of the KIX8 and KIX9 genes, which results in plants having a higher yield.

### BACKGROUND

Much of the economic value of crop plants comes either directly or indirectly from the growth and form of the lateral shoot organs: the leaves, the flowers and the fruit/seeds/seed pods that develop from the flowers. Some of the economic value derived from variation in plant organ size or shape is obvious. Larger fruit or seed may represent a greater harvest index, more grain or fruit per plant or per crop area. Other economic impacts of variability in organ size and shape are indirect. For example, the leaf blade is the main site of photosynthesis and respiration in higher plants and leaf size and shape are key factors influencing these processes. Larger leaves on short stems can result in a higher grain yield, due to more carbon being made available for seed development instead of vegetative growth. The economic value of differences in the size and shape of plant organs are many. By 2050, the world population is likely to be 9.1 billion and there is a need to increase crop production by approximately 50% or more by 2050 without extra land. There is, therefore, a need to identify novel pathways and genes in plants, which modulation results in higher yields. This disclosure satisfies this need, in particular, since the disclosure relates to the combined down-regulation of two genes, i.e., KIX8 and KIX9, in plants resulting in a 30% increased leaf growth.

### BRIEF SUMMARY

In one aspect, the disclosure provides a dicotyledonous plant having an at least 80% reduction in the expression of the KIX8 gene and the KIX9 gene.

In another aspect, the disclosure provides a dicotyledonous plant having a loss of function of the KIX8 and KIX9 genes.

In yet another aspect, the disclosure provides a dicotyledonous plant according to which has a gene disruption in KIX8 and KIX9 and does not express the KIX8 and KIX9 genes.

In yet another aspect, the disclosure provides for a seed or a plant cell derived from a plant having an at least 80% reduction in the expression of the KIX8 and the KIX9 gene.

In yet another aspect, the disclosure provides for a seed or a plant cell derived from a plant having a loss of function of the KIX8 and KIX9 genes.

In still another aspect, the disclosure provides a dicotyledonous plant according to which has a gene disruption in KIX8 and KIX9 and does not express the KIX8 and KIX9 genes.

In yet another aspect, the disclosure provides for a method to produce a dicotyledonous plant having an at least 80% reduction in the expression of the KIX8 and KIX9 genes comprising the introduction of a silencing RNA construct directed to KIX8 and KIX9 or an artificial microRNA directed to KIX8 and KIX9.

In yet another aspect, the disclosure provides for a method to produce a dicotyledonous plant having an at least 30% increased yield comprising i) the introduction of a silencing RNA construct directed to KIX8 and KIX9 or ii) an artificial microRNA directed to KIX8 and KIX9.

In yet another aspect, the disclosure provides a recombinant vector comprising a silencing RNA construct directed to KIX8 and KIX9 or an artificial microRNA directed to KIX8 and KIX9.

In yet another aspect, the disclosure provides a dicotyledonous plant or plant cell or plant seed comprising a recombinant vector comprising a silencing RNA construct directed to KIX8 and KIX9 or an artificial microRNA directed to KIX8 and KIX9.

### BRIEF DESCRIPTION OF THE DRAWINGS

FIGS. 1A-1F: 35S:ami-ppd rosette, leaf and cellular phenotype. FIG. 1A, wild-type (left) and 35S::ami-ppd (right) plants grown for 25 days after stratification (“DAS”) in soil; FIG. 1B, area of individual leaves of wild-type and 35S::ami-ppd plants grown in vitro for 21 DAS; FIG. 1C, leaf area; FIG. 1D, cell number; FIG. 1E, cell area over time of 35S::ami-ppd leaf 1-2 compared to wild-type; and FIG. 1F, cell size distribution (N=3; *: p value<0.05).

FIG. 2: PPD2 DNA binding sites identified by TCHAP-seq by using a cell culture expressing 35S::PPD2-HBH. Panel A, genome-wide distribution of PPD2 DNA binding sites (peaks identified with MACS (Zhang et al., 2008)) in function of the gene structure (intergenic and UTR 5′, coding region and introns, intergenic and UTR 3′). Panel B, GenomeView representation (Abeel et al., 2012) of the TChAP-seq results for PPD1, PPD2 and DFL1 in the 35SS::PPD2-HBH and the control (35S::HBH) cell culture. Forward reads are represented in green, reverse reads in blue and total coverage in yellow. The coding regions are represented by the black arrow.

FIGS. 3A and 3B: Direct target genes of PPD2 differentially expressed in 35S::ami-ppd growing leaves. FIG. 3A, overlap between the genes identified by TCHAP-seq and the genes differentially expressed in the growing leaves (13 DAS) of 35S::ami-ppd line.

FIG. 3B, time course analysis of PPD2 target gene expression after induction of PPD2 in 35S::PPD2 GR line treated or non-treated with DEX (Dexamethasone) (N=3, * pvalue<0.05).

FIG. 4: The KIX proteins interact with the PPD domain through a KIX domain and are TPL (TOPLESS) adaptors. Panel A, schematic overview of the KIX1 protein structure; Panel B, representative confocal microscopy image of an Arabidopsis root cell, expressing KIX1-GFP; Panel C, Y2H interaction analysis of both PPD and KIX proteins; Panel D, truncations of PPD2 were tested to identify the KIX1 interaction domain; Panel E, KIX1 and KIX2 interaction with TF from different protein families was confirmed; Panel F, direct interaction with TPL and the necessity of the EAR domain was tested by Y2H; Panel G, yeast three-hybrid experiment to test bridging of PPD-TPL interaction through KIX; Panel H, GAL4DBD fused KIX1 and KIX2 proteins were tested for transcriptional repression activity of the UAS:fLUC reporter in Tobacco BY-2 protoplasts. Y2H and Y3H assays were performed. For Y3H, KIX was expressed in addition using the vector pMG426 and transformants were selected on medium lacking Leu and Trp and Ura (-3) or selective medium additionally lacking His (-4).

FIG. 5: Rosette and leaf phenotype of the kix8, kix9 and kix8-kix9 mutants. Panel A, from top to bottom: wild-type and kix8, kix9 and kix8-kix9 and 35S::ami-ppd plants grown for 25 DAS in soil. Panel B, area of individual leaves of wild-type, kix8, kix9 and kix8-kix9 and 35S::ami-ppd plants grown in soil for 21 DAS (*: p-value<0.05).

FIGS. 6A-6C: KIX 8 and KIX9 are important for the regulation of the expression of PPD2 target genes. FIGS. 6A and 6B: Time course analysis of PPD2 target gene expression in wild-type, kix8, kix9 and kix8-kix9 leaf 1-2. (N=3, *: p-value<0.05). FIG. 6C: PPD2- and KIX8-dependent activation of the promoters of CYCD3;2 and CYCD3;3 by the protoplast activation assay. Indicated values are luciferase detection levels. (a, b, c represent significantly different values compared to control (a), PPD2 (b), and KIX8 (c). Error bars indicate sd).

### DETAILED DESCRIPTION

To facilitate the understanding of this disclosure, a number of terms are defined below. Terms defined herein (unless otherwise specified) have meanings as commonly understood by a person of ordinary skill in the areas relevant to the this disclosure. As used in this specification and its appended claims, terms such as “a,” “an” and “the” are not intended to refer to only a singular entity, but include the general class of which a specific example may be used for illustration, unless the context dictates otherwise. The terminology herein is used to describe specific embodiments of the disclosure, but their usage does not delimit the disclosure, except as outlined in the claims.

This disclosure is derived from the unexpected findings that a combined down-regulation of the expression of two genes (i.e., KIX8 and KIX9) leads to a 30% increase in plant yield.

The KIX8 gene is sometimes also referred to in the art as the KIX2 gene or the NIP2 gene. For ease of reference and avoidance of doubt, a representative of the KIX8 gene is represented by SEQ ID NO:1 (as derived from Arabidopsis thaliana).

Thus, a representative of the plant KIX8 gene in Arabidopsis is AT3G24150 (TAIR accession, www.arabidopsis.org), which is the KIX8 gene for which the genome sequence is depicted in SEQ ID NO:1 and its protein coding sequence is depicted in SEQ ID NO:2.

The KIX9 gene is sometimes referred to in the art also as the KIX1 gene or the NIP1 gene.

Thus, a representative of the plant KIX9 gene in Arabidopsis is AT4G32295 (TAIR accession, www.arabidopsis.org), which is the KIX9 gene for which the genome sequence is depicted in SEQ ID NO:3 and its protein coding sequence is depicted in SEQ ID NO:4.

In this disclosure, the meaning of “KIX8 gene” refers to SEQ ID NO:1 or SEQ ID NO:2 or a plant orthologous gene derived thereof. The “KIX9 gene” refers to SEQ ID NO:3 or SEQ ID NO:4 or a plant orthologous gene derived thereof.

Without limiting the disclosure to a particular mechanism, it is envisaged that the combined down-regulation of the KIX8 gene and the KIX9 gene leads to an increased plant yield.

A plant orthologous KIX8 or a plant orthologous KIX9 gene (as defined herein) is an orthologous gene of SEQ ID NO:1, 2, 3 or 4 and derived from a dicotyledonous plant.

The term “plant yield” as used herein generally refers to a measurable product from a plant, particularly a crop. Yield and yield increase (in comparison to a non-transformed starting plant or mutant plant or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeably. As used herein, the term “improved yield” or the term “increased yield” means any improvement in the yield of any measured plant product, such as grain, fruit, leaf, root, or fiber. In accordance with the disclosure, changes in different phenotypic traits may improve yield. For example, and without limitation, parameters such as floral organ development, root initiation, root biomass, seed number, seed weight, harvest index, leaf formation, phototropism, apical dominance, and fruit development, are suitable measurements of improved yield. Increased yield includes higher fruit yields, higher seed yields, higher fresh matter production, and/or higher dry matter production. Any increase in yield is an improved yield in accordance with the disclosure. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bu/acre yield of soybeans derived from a crop comprising plants that are transgenic for the chimeric genes of the disclosure or mutants of the KIX8/KIX9 genes as compared with the bu/acre yield from untransformed soybeans cultivated under the same conditions, is an improved yield in accordance with the disclosure.

The increased or improved yield can be achieved in the absence or presence of stress conditions. For example, enhanced or increased “yield” refers to one or more yield parameters selected from the group consisting of: biomass yield; dry biomass yield; aerial dry biomass yield; underground dry biomass yield; fresh-weight biomass yield; aerial fresh-weight biomass yield; underground fresh-weight biomass yield; enhanced yield of harvestable parts, either dry or fresh-weight or both, either aerial or underground or both; enhanced yield of crop fruit, either dry or fresh-weight or both, either aerial or underground or both; and enhanced yield of seeds, either dry or fresh-weight or both, either aerial or underground or both.

“Crop yield” is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant.

The yield of a plant can depend on the specific plant/crop of interest as well as its intended application (such as food production, feed production, processed food production, biofuel, biogas or alcohol production, or the like) of interest in each particular case. Thus, in one embodiment, yield can be calculated as harvest index (expressed as a ratio of the weight of the respective harvestable parts divided by the total biomass), weight of harvestable parts per area (acre, square meter, or the like), and the like. The harvest index is the ratio of yield biomass to the total cumulative biomass at harvest. Harvest index is relatively stable under many environmental conditions, and so a robust correlation between plant size and grain yield is possible.

Measurements of plant size in early development, under standardized conditions in a growth chamber or greenhouse, are standard practices to measure potential yield advantages conferred by the presence of a transgene. Accordingly, the yield of a plant can be increased by improving one or more of the yield-related phenotypes or traits. Such yield-related phenotypes or traits of a plant, the improvement of which results in increased yield, comprise, without limitation, the increase of the intrinsic yield capacity of a plant, improved nutrient use efficiency, and/or increased stress tolerance. For example, “yield” refers to biomass yield, e.g., to dry weight biomass yield and/or fresh-weight biomass yield. “Biomass yield” refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry-weight or a moisture-adjusted basis. Biomass yield may be calculated on a per plant basis or in relation to a specific area (e.g., biomass yield per acre/square meter/or the like).

“Yield” can also refer to seed yield, which can be measured by one or more of the following parameters: number of seeds or number of filled seeds (per plant or per area (acre/square meter/or the like)); seed filling rate (ratio between number of filled seeds and total number of seeds); number of flowers per plant; seed biomass or total seeds weight (per plant or per area (acre/square meter/or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters for measuring seed yield are also known in the art. Seed yield may be determined on a dry-weight or on a fresh-weight basis, or typically on a moisture-adjusted basis, e.g., at 15.5% moisture. For example, the term “increased yield” means that a plant exhibits an increased growth rate, e.g., in the absence or presence of abiotic environmental stress, compared to the corresponding wild-type plant.

An increased growth rate may confer or be reflected inter alia by an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds.

A prolonged growth comprises survival and/or continued growth of the plant at the moment when the non-transformed wild-type organism shows visual symptoms of deficiency and/or death. When the plant of the disclosure is a soy plant, increased yield for soy plants means increased seed yield, in particular, for soy varieties used for feed or food. Increased seed yield of soy refers, for example, to an increased kernel size or weight, an increased kernel per pod, or increased pods per plant. When the plant of the disclosure is an oil seed rape (OSR) plant, increased yield for OSR plants means increased seed yield, in particular, for OSR varieties used for feed or food. Increased seed yield of OSR refers to an increased seed size or weight, an increased seed number per silique, or increased siliques per plant. When the plant of the disclosure is a cotton plant, increased yield for cotton plants means increased lint yield. Increased lint yield of cotton refers in one embodiment to an increased length of lint.

Increased yield can typically be achieved by enhancing or improving one or more yield-related traits of the plant. Such yield-related traits of a plant comprise, without limitation, the increase of the intrinsic yield capacity of a plant, improved nutrient use efficiency, and/or increased stress tolerance, in particular, increased abiotic stress tolerance. Intrinsic yield capacity of a plant can be, for example, manifested by improving the specific (intrinsic) seed yield (e.g., in terms of increased seed/grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigor/early vigor, enhanced efficiency of germination (under-stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

Any relevant method known in the art can be adapted to reduce or eliminate the combined activity of a plant KIX8 gene and KIX9 gene (hereinafter, the combination of KIX8 and KIX9 is abbreviated as “KIX8/KIX9” and can be used as singular or plural in the description but it is always meant to mean a combination of KIX8 and KIX9) and can be used to increase plant yield, in particular, to increase plant biomass. In some embodiments, a polynucleotide is introduced into a plant that may inhibit the expression of a KIX8/KIX9 polypeptide directly, by preventing transcription or translation of a KIX8/KIX9 messenger RNA, or indirectly, by encoding a polypeptide that inhibits the transcription or translation of a KIX8/KIX9 gene encoding a KIX8/KIX9 polypeptide. Methods for inhibiting or eliminating the expression of a gene in a plant are well known in the art, and any such method may be used in this disclosure to inhibit the expression of the KIX8/KIX9 polypeptide. In other embodiments, a polynucleotide that encodes a polypeptide that inhibits the activity of a KIX8/KIX9 polypeptide is introduced into a plant. In yet other embodiments, the activity of a KIX8/KIX9 is inhibited through disruption of a KIX8/KIX9 gene. Many methods may be used to reduce or eliminate the activity of a KIX8/KIX9 polypeptide. In addition, more than one method may be used to reduce the activity of a single KIX8/KIX9 polypeptide. In some embodiments, the KIX8/KIX9 activity is reduced through the disruption of the KIX8/KIX9 gene or a reduction in the expression of the KIX8/KIX9 gene. A KIX8/KIX9 gene can comprise, e.g., at least about 35%, 40%, 45%, 50%, 55%, 60%, 65%, 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.5% or more sequence identity to SEQ ID NO:1, 2, 3 or 4. Many KIX8/KIX9 genes are known to those of skill in the art and are readily available through sources such as GENBANK and the like. The expression of any KIX8/KIX9 gene may be reduced according to methods described in the disclosure.

In accordance with this disclosure, the expression of a KIX8/KIX9 is inhibited if the transcript or protein level of the KIX8/KIX9 is statistically lower than the transcript or protein level of the same KIX8/KIX9 in a plant that has not been genetically modified or mutagenized to inhibit the expression of that KIX8/KIX9. In particular embodiments of the disclosure, the transcript or protein level of the KIX8/KIX9 in a modified plant according to the disclosure is less than 95%, less than 90%, less than 85%, less than 80%, less than 75%, less than 70%, less than 60%, less than 50%, less than 40%, less than 30%, less than 20%, less than 10%, or less than 5% of the protein level of the same KIX8/KIX9 in a plant that is not a mutant or that has not been genetically modified to inhibit the expression of that KIX8/KIX9. The expression level of the KIX8/KIX9 may be measured directly, for example, by assaying for the level of KIX8/KIX9 expressed in the cell or plant, or indirectly, for example, by measuring the KIX8/KIX9 activity in the cell or plant. The activity of a KIX8/KIX9 protein is “eliminated,” according to the disclosure, when it is not detectable by at least one assay method. Methods for assessing KIX8/KIX9 activity are known in the art and include measuring levels of KIX8/KIX9, which can be recovered and assayed from cell extracts.

In other embodiments of the disclosure, the activity of KIX8/KIX9 is reduced or eliminated by transforming a plant cell with an expression cassette comprising a polynucleotide encoding a polypeptide that inhibits the activity of KIX8/KIX9. The activity of a KIX8/KIX9 is inhibited according to this disclosure if the activity of that KIX8/KIX9 in the transformed plant or cell is statistically lower than the activity of that KIX8/KIX9 in a plant that has not been genetically modified to inhibit the activity of at least one KIX8/KIX9. In particular embodiments of the disclosure, a KIX8/KIX9 activity of a modified plant according to the disclosure is less than 95%, less than 90%, less than 85%, less than 80%, less than 75%, less than 70%, less than 60%, less than 50%, less than 40%, less than 30%, less than 20%, less than 10%, or less than 5% of that KIX8/KIX9 activity in an appropriate control plant that has not been genetically modified to inhibit the expression or activity of the KIX8/KIX9.

In other embodiments, the activity of a KIX8/KIX9 protein may be reduced or eliminated by disrupting the genes encoding the KIX8/KIX9. The disruption inhibits expression or activity of KIX8/KIX9 protein compared to a corresponding control plant cell lacking the disruption. In one embodiment, the endogenous KIX8/KIX9 gene comprises two or more endogenous KIX8/KIX9 genes. Similarly, in another embodiment, in particular plants, the endogenous KIX8/KIX9 gene comprises three or more endogenous KIX8/KIX9 genes. The wording “two or more endogenous KIX8/KIX9 genes” or “three or more endogenous KIX8/KIX9 genes” refers to two or more or three or more homologs of KIX8/KIX9 or KIX8/KIX9 2 but it is not excluded that two or more or three or more combinations of homologs of KIX8/KIX9 1 or KIX8/KIX9 2 are disrupted (or their activity reduced).

In another embodiment, the disruption step comprises insertion of one or more transposons, where the one or more transposons are inserted into the endogenous KIX8/KIX9 gene. In yet another embodiment, the disruption comprises one or more point mutations in the endogenous KIX8/KIX9 gene. The disruption can be a homozygous disruption in the KIX8/KIX9 gene. Alternatively, the disruption is a heterozygous disruption in the KIX8/KIX9 gene. In certain embodiments, when more than one KIX8/KIX9 gene is involved, there is more than one disruption, which can include homozygous disruptions, heterozygous disruptions or a combination of homozygous disruptions and heterozygous disruptions.

Detection of expression products is performed either qualitatively (by detecting presence or absence of one or more products of interest) or quantitatively (by monitoring the level of expression of one or more products of interest). In one embodiment, the expression product is an RNA expression product. Aspects of the disclosure optionally include monitoring an expression level of a nucleic acid, polypeptide as noted herein for detection of KIX8/KIX9 or measuring the amount of yield increase in a plant or in a population of plants.

Thus, many methods may be used to reduce or eliminate the activity of a KIX8/KIX9 gene. More than one method may be used to reduce the activity of a single plant KIX8/KIX9 gene combination. In addition, combinations of methods may be employed to reduce or eliminate the activity of two or more different KIX8/KIX9 gene combinations. Non-limiting examples of methods of reducing or eliminating the expression of a plant KIX8/KIX9 are given below.

In some embodiments of this disclosure, a polynucleotide is introduced into a plant that, upon introduction or expression, inhibits the expression of a KIX8/KIX9 gene of the disclosure. The term “expression” as used herein refers to the biosynthesis of a gene product, including the transcription and/or translation of the gene product. For example, for the purposes of this disclosure, an expression cassette capable of expressing a polynucleotide that inhibits the expression of at least one KIX8/KIX9 polypeptide is an expression cassette capable of producing an RNA molecule that inhibits the transcription and/or translation of at least one KIX8/KIX9 polypeptide of the disclosure. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide. Further, “expression” of a gene can refer to the transcription of the gene into a non-protein coding transcript.

As used herein, “polynucleotide” includes reference to a deoxyribopolynucleotide, ribopolynucleotide, or analogs thereof, that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide” as it is employed herein embraces such chemically, enzymatically or metabolically modified forms of polynucleotides, as well as the chemical forms of DNA and RNA characteristic of viruses and cells, including inter alia, simple and complex cells.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “encoding” or “encoded,” with respect to a specified nucleic acid, is meant comprising the information for transcription into an RNA and in some embodiments, translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code.

Examples of polynucleotides that inhibit the expression of a KIX8/KIX9 polypeptide are given below. In some embodiments of the disclosure, inhibition of the expression of a KIX8/KIX9 polypeptide may be obtained by sense suppression or co-suppression. For co-suppression, an expression cassette is designed to express an RNA molecule corresponding to all or part of a messenger RNA encoding a KIX8/KIX9 polypeptide in the “sense” orientation. Over-expression of the RNA molecule can result in reduced expression of the native gene. Accordingly, multiple plant lines transformed with the co-suppression expression cassette are screened to identify those that show the greatest inhibition of KIX8/KIX9 polypeptide expression.

The polynucleotide used for co-suppression may correspond to all or part of the sequence encoding the KIX8/KIX9 polypeptide, all or part of the 5′ and/or 3′ untranslated regions of a KIX8/KIX9 polypeptide transcript or all or part of both the coding sequence and the untranslated regions of a transcript encoding a KIX8/KIX9 polypeptide. A polynucleotide used for co-suppression or other gene-silencing methods may share 99%, 98%, 97%, 96%, 95%, 94%, 93%, 92%, 91%, 90%, 89%, 88%, 8′7%, 85%, 80%, or less sequence identity with the target sequence. When portions of the polynucleotides are used to disrupt the expression of the target gene, generally, sequences of at least 15, 19, 20, 21, 22, 23, 24, 25, 26, 27, 28, 29, 30, 35, 40, 45, 50, 60, 70, 80, 90, 100, 200, 300, 400, 450, 500, 550, 600, 650, 700, 750, 800, 900, or 1000 contiguous nucleotides or greater may be used. In some embodiments where the polynucleotide comprises all or part of the coding region for the KIX8/KIX9 polypeptide, the expression cassette is designed to eliminate the start codon of the polynucleotide so that no protein product will be translated.

Co-suppression may be used to inhibit the expression of plant genes to produce plants having undetectable protein levels for the proteins encoded by these genes. See, for example, Broin, et al. (2002), Plant Cell 14:1417-1432. Co-suppression may also be used to inhibit the expression of multiple proteins in the same plant. See, for example, U.S. Pat. No. 5,942,657. Methods for using co-suppression to inhibit the expression of endogenous genes in plants are described in U.S. Pat. Nos. 5,034,323, 5,283,184 and 5,942,657, each of which is incorporated herein by this reference. The efficiency of co-suppression may be increased by including a poly-dT region in the expression cassette at a position 3′ to the sense sequence and 5′ of the polyadenylation signal. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323, incorporated herein by this reference.

In some embodiments of the disclosure, inhibition of the expression of the KIX8/KIX9 polypeptide may be obtained by antisense suppression. For antisense suppression, the expression cassette is designed to express an RNA molecule complementary to all or part of a messenger RNA encoding the KIX8/KIX9 polypeptide. Over-expression of the antisense RNA molecule can result in reduced expression of the native gene. Accordingly, multiple plant lines transformed with the antisense suppression expression cassette are screened to identify those that show the greatest inhibition of KIX8/KIX9 polypeptide expression.

The polynucleotide for use in antisense suppression may correspond to all or part of the complement of the sequence encoding the KIX8/KIX9 polypeptide, all or part of the complement of the 5′ and/or 3′ untranslated region of the KIX8/KIX9 transcript, or all or part of the complement of both the coding sequence and the untranslated regions of a transcript encoding the KIX8/KIX9 polypeptide.

In addition, the antisense polynucleotide may be fully complementary (i.e., 100% identical to the complement of the target sequence) or partially complementary (i.e., less than 100%, including, but not limited to, 99%, 98%, 97%, 96%, 95%, 94%, 93%, 92%, 91%, 90%, 89%, 88%, 87%, 85%, 80%, identical to the complement of the target sequence, which in some embodiments is SEQ ID NOS:1, 2, 3, 4 or a plant orthologous to the gene sequence thereof) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used.

Methods for using antisense suppression to inhibit the expression of endogenous genes in plants are described, for example, in U.S. Pat. No. 5,759,829, which is incorporated herein by this reference. Efficiency of antisense suppression may be increased by including a poly-dT region in the expression cassette at a position 3′ to the antisense sequence and 5′ of the polyadenylation signal.

In some embodiments of the disclosure, inhibition of the expression of a KIX8/KIX9 polypeptide may be obtained by double-stranded RNA (dsRNA) interference. For dsRNA interference, a sense RNA molecule like that described above for co-suppression and an antisense RNA molecule that is fully or partially complementary to the sense RNA molecule are expressed in the same cell, resulting in inhibition of the expression of the corresponding endogenous messenger RNA. Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of KIX8/KIX9 polypeptide expression. Methods for using dsRNA interference to inhibit the expression of endogenous plant genes are described in WO9949029, WO9953050, WO9961631 and WO0049035, each of which is incorporated herein by this reference.

In some embodiments of the disclosure, inhibition of the expression of a KIX8/KIX9 polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell (2003), Nat. Rev. Genet. 4:29-38 and the references cited therein. For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. The antisense sequence may be located “upstream” of the sense sequence (i.e., the antisense sequence may be closer to the promoter-driving expression of the hairpin RNA than the sense sequence). The base-paired stem region may correspond to a portion of a promoter sequence controlling expression of the gene to be inhibited. A polynucleotide designed to express an RNA molecule having a hairpin structure comprises a first nucleotide sequence and a second nucleotide sequence that is the complement of the first nucleotide sequence, and wherein the second nucleotide sequence is in an inverted orientation relative to the first nucleotide sequence. Thus, the base-paired stem region of the molecule generally determines the specificity of the RNA interference.

The sense sequence and the antisense sequence are generally of similar lengths but may differ in length. Thus, these sequences may be portions or fragments of at least 10, 19, 20, 21, 22, 23, 24, 25, 26, 27, 28, 29, 30, 50, 70, 90, 100, 120, 140, 160, 180, 200, 220, 240, 260, 280, 300, 320, 340, 360, 380, 400, 500, 600, 700, 800, 900 nucleotides in length, or at least 1, 2, 3, 4, 5, 6, 7, 8, 9, or 10 kb in length. The loop region of the expression cassette may vary in length. Thus, the loop region may be at least 10, 20, 30, 40, 50, 75, 100, 200, 300, 400, 500, 600, 700, 800, 900 nucleotides in length, or at least 1, 2, 3, 4, 5, 6, 7, 8, 9, or 10 kb in length. hpRNA molecules are highly efficient at inhibiting the expression of endogenous genes and the RNA interference they induce is inherited by subsequent generations of plants. See, for example, Waterhouse and Helliwell (2003), Nat. Rev. Genet. 4:29-38.

A transient assay for the efficiency of hpRNA constructs to silence gene expression in vivo has been described by Panstruga et al. (2003), Mol. Biol. Rep. 30:135-140, incorporated herein by this reference. For ihpRNA, the interfering molecules have the same general structure as for hpRNA, but the RNA molecule additionally comprises an intron in the loop of the hairpin that is capable of being spliced in the cell in which the ihpRNA is expressed. The use of an intron minimizes the size of the loop in the hairpin RNA molecule following splicing, and this increases the efficiency of interference. See, for example, Smith et al. (2000), Nature 407:319-320. In fact, Smith et al., show 100% suppression of endogenous gene expression using ihpRNA-mediated interference. In some embodiments, the intron is the ADH1 intron 1. Methods for using ihpRNA interference to inhibit the expression of endogenous plant genes are described, for example, in Smith et al. (2000), Nature 407:319-320; Waterhouse and Helliwell (2003), Nat. Rev. Genet. 4:29-38; Helliwell and Waterhouse (2003), Methods 30:289-295; and U.S. 2003/180945, each of which is incorporated herein by this reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO0200904 incorporated herein by this reference.

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the KIX8/KIX9 polypeptide). Methods of using amplicons to inhibit the expression of endogenous plant genes are described, for example, in U.S. Pat. No. 6,635,805, which is incorporated herein by this reference.

In some embodiments, the polynucleotide expressed by the expression cassette of the disclosure is catalytic RNA or has ribozyme activity specific for the messenger RNA of the KIX8/KIX9 polypeptide. Thus, the polynucleotide causes the degradation of the endogenous messenger RNA, resulting in reduced expression of the KIX8/KIX9 polypeptide. This method is described, for example, in U.S. Pat. No. 4,987,071, incorporated herein by this reference. In some embodiments of the disclosure, inhibition of the expression of a KIX8/KIX9 polypeptide may be obtained by RNA interference by expression of a polynucleotide encoding a micro RNA (miRNA). miRNAs are regulatory agents consisting of about 22 ribonucleotides. miRNA are highly efficient at inhibiting the expression of endogenous genes. See, for example, Javier et al. (2003), Nature 425:257-263, incorporated herein by this reference.

For miRNA interference, the expression cassette is designed to express an RNA molecule that is modeled on an endogenous pre-miRNA gene wherein the endogenous miRNA and miRNA* sequence are replaced by sequences targeting the KIX8/KIX9 mRNA. The miRNA gene encodes an RNA that forms a hairpin structure containing a 22-nucleotide sequence that is complementary to another endogenous gene (target sequence). For suppression of the KIX8/KIX9, the 22-nucleotide sequence is selected from a KIX8/KIX9 transcript sequence and contains 22 nucleotides of the KIX8/KIX9 in sense orientation (the miRNA* sequence) and 21 nucleotides of a corresponding antisense sequence that is complementary to the sense sequence and complementary to the target mRNA (the miRNA sequence). No perfect complementarity between the miRNA and its target is required, but some mismatches are allowed. Up to four mismatches between the miRNA and miRNA* sequence are also allowed. miRNA molecules are highly efficient at inhibiting the expression of endogenous genes, and the RNA interference they induce is inherited by subsequent generations of plants.

In some embodiments, polypeptides or polynucleotide-encoding polypeptides can be introduced into a plant, wherein the polypeptide is capable of inhibiting the activity of a KIX8/KIX9 polypeptide. The terms “polypeptide,” “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

The terms “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass known analogs of natural amino acids that can function in a similar manner as naturally occurring amino acids.

Significant advances have been made in the last few years toward development of methods and compositions to target and cleave genomic DNA by site-specific nucleases (e.g., Zinc Finger Nucleases (ZFNs), Meganucleases, Transcription Activator-Like Effector Nucelases (TALENS) and Clustered Regularly Interspaced Short Palindromic Repeats/CRISPR-associated nuclease (CRISPR/Cas) with an engineered crRNA/tracr RNA), to induce targeted mutagenesis, induce targeted deletions of cellular DNA sequences, and facilitate targeted recombination of an exogenous donor DNA polynucleotide within a predetermined genomic locus. See, for example, U.S. Patent Publication Nos. 2003/0232410, 2005/0208489, 2005/0026157, 2005/0064474, and 2006/0188987, and International Patent Publication No. WO 2007/014275, the disclosures of each of which are incorporated herein by this reference in their entireties for all purposes. U.S. Patent Publication No. 2008/0182332 describes use of non-canonical zinc finger nucleases (ZFNs) for targeted modification of plant genomes and U.S. Patent Publication No. 2009/0205083 describes ZFN-mediated targeted modification of a plant EPSPs genomic locus. Current methods for targeted insertion of exogenous DNA typically involve co-transformation of plant tissue with a donor DNA polynucleotide containing at least one transgene and a site-specific nuclease (e.g., ZFN) which is designed to bind and cleave a specific genomic locus of an actively transcribed coding sequence. This causes the donor DNA polynucleotide to stably insert within the cleaved genomic locus resulting in targeted gene addition at a specified genomic locus comprising an actively transcribed coding sequence.

As used herein, the term “zinc fingers” defines regions of amino acid sequence within a DNA binding protein binding domain whose structure is stabilized through coordination of a zinc ion.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; 6,534,261; and 6,794,136; see also WO 98/53058; WO 98/53059; WO 98/53060; WO 02/016536 and WO 03/016496.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Patent Publication No. 20110301073, incorporated herein by this reference in its entirety.

The CRISPR (Clustered Regularly Interspaced Short Palindromic Repeats)/Cas (CRISPR Associated) nuclease system. Briefly, a “CRISPR DNA binding domain” is a short-stranded RNA molecule that acting in concert with the CAS enzyme can selectively recognize, bind, and cleave genomic DNA. The CRISPR/Cas system can be engineered to create a double-stranded break (DSB) at a desired target in a genome, and repair of the DSB can be influenced by the use of repair inhibitors to cause an increase in error-prone repair. See, e.g., Jinek et al. (2012) Science 337:816-821; Jinek et al. (2013), eLife 2:e00471, and David Segal (2013), eLife 2:e00563).

Zinc finger, CRISPR and TALE binding domains can be “engineered” to bind to a predetermined nucleotide sequence, for example, via engineering (altering one or more amino acids) of the recognition helix region of a naturally occurring zinc finger. Similarly, TALEs can be “engineered” to bind to a predetermined nucleotide sequence, for example, by engineering of the amino acids involved in DNA binding (the repeat variable diresidue or RVD region). Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also, WO 98/53058; WO 98/53059; WO 98/53060; WO 02/016536 and WO 03/016496 and U.S. Publication Nos. 20110301073, 20110239315 and 20119145940.

A “selected” zinc finger protein, CRISPR or TALE is a protein not found in nature whose production results primarily from an empirical process such as phage display, interaction trap or hybrid selection. See, e.g., U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970; WO 01/88197; and WO 02/099084 and U.S. Publication Nos. 20110301073, 20110239315 and 20119145940.

In one embodiment, the polynucleotide encodes a zinc finger protein that binds to a gene encoding a KIX8/KIX9 polypeptide, resulting in reduced expression of the gene. In particular embodiments, the zinc finger protein binds to a regulatory region of a KIX8/KIX9. In other embodiments, the zinc finger protein binds to a messenger RNA encoding a KIX8/KIX9 polypeptide and prevents its translation. Methods of selecting sites for targeting by zinc finger proteins have been described, for example, in U.S. Pat. No. 6,453,242, and methods for using zinc finger proteins to inhibit the expression of genes in plants are described, for example, in US2003/0037355, each of which is incorporated herein by this reference.

In another embodiment, the polynucleotide encoded a TALE protein that binds to a gene encoding a KIX8/KIX9 polypeptide, resulting in reduced expression of the gene. In particular embodiments, the TALE protein binds to a regulatory region of a KIX8/KIX9. In other embodiments, the TALE protein binds to a messenger RNA encoding a KIX8/KIX9 polypeptide and prevents its translation. Methods of selecting sites for targeting by zinc finger proteins have been described in, e.g., M. J. Moscou and A. J. Bogdanove (2009, a simple cipher governs DNA recognition by TAL effectors, Science 326:1501) and R., Morbitzer, P. Romer, J. Boch, and T. Lahaye (2010, regulation of selected genome loci using de novo-engineered transcription activator-like effector (TALE)-type transcription factors, Proc. Natl. Acad. Sci. U.S.A. 107:21617-21622).

In some embodiments of the disclosure, the polynucleotide encodes an antibody that binds to at least one KIX8/KIX9 polypeptide and reduces the activity of the KIX8/KIX9 polypeptide. In another embodiment, the binding of the antibody results in increased turnover of the antibody-KIX8/KIX9 complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

In some embodiments of this disclosure, the activity of a KIX8/KIX9 is reduced or eliminated by disrupting the gene encoding the KIX8/KIX9 polypeptide. The gene encoding the KIX8/KIX9 polypeptide may be disrupted by any method known in the art. For example, in one embodiment, the gene is disrupted by transposon tagging. In another embodiment, the gene is disrupted by mutagenizing plants using random or targeted mutagenesis and screening for plants that have an increased yield.

In one embodiment of the disclosure, transposon tagging is used to reduce or eliminate the KIX8/KIX9 activity of one or more KIX8/KIX9 polypeptides. Transposon tagging comprises inserting a transposon within an endogenous KIX8/KIX9 gene to reduce or eliminate expression of the KIX8/KIX9 polypeptide. In this embodiment, the expression of one or more KIX8/KIX9 polypeptides is reduced or eliminated by inserting a transposon within a regulatory region or coding region of the gene encoding the KIX8/KIX9 polypeptide. A transposon that is within an exon, intron, 5′ or 3′ untranslated sequence, a promoter or any other regulatory sequence of a KIX8/KIX9 gene may be used to reduce or eliminate the expression and/or activity of the encoded KIX8/KIX9 polypeptide.

Methods for the transposon tagging of specific genes in plants are well known in the art. See, for example, Meissner, et al. (2000) Plant J. 22:265-21. In addition, the TUSC process for selecting Mu insertions in selected genes has been described in U.S. Pat. No. 5,962,764, which is incorporated herein by this reference.

Additional methods for decreasing or eliminating the expression of endogenous genes in plants are also known in the art and can be similarly applied to this disclosure. These methods include other forms of mutagenesis, such as ethyl methanesulfonate-induced mutagenesis, deletion mutagenesis and fast neutron deletion mutagenesis used in a reverse genetics sense (with PCR) to identify plant lines in which the endogenous gene has been deleted. For examples of these methods, see, Ohshima, et al. (1998) Virology 243:472-481; Okubara, et al. (1994) Genetics 137:867-874; and Quesada, et al. (2000) Genetics 154:421-436, each of which is incorporated herein by this reference. In addition, a fast and automatable method for screening for chemically induced mutations, TILLING (Targeting Induced Local Lesions in Genomes), using denaturing HPLC or selective endonuclease digestion of selected PCR products is also applicable to this disclosure. See, McCallum, et al. (2000) Nat. Biotechnol. 18:455-457, incorporated herein by this reference. Mutations that impact gene expression or that interfere with the function of the encoded protein are well known in the art. Insertional mutations in gene exons usually result in null mutants. Mutations in conserved residues are particularly effective in inhibiting the activity of the encoded protein. Conserved residues of plant KIX8/KIX9 polypeptides suitable for mutagenesis with the goal to eliminate KIX8/KIX9 activity have been described. Such mutants can be isolated according to well-known procedures, and mutations in different KIX8/KIX9 loci can be stacked by genetic crossing. See, for example, Gruis, et al. (2002) Plant Cell 14:2863-2882. In another embodiment of this disclosure, dominant mutants can be used to trigger RNA silencing due to gene inversion and recombination of a duplicated gene locus. See, for example, Kusaba, et al. (2003) Plant Cell 15:1455-1467.

Single-stranded DNA can also be used to down-regulate the expression of KIX8/KIX9 genes. Methods for gene suppression using ssDNA are, e.g., described in WO 2011/112570.

In yet another embodiment, protein interference as described in the patent application WO2007/071789 (means and methods for mediating protein interference) can be used to down-regulate a gene product. The latter technology is a knock-down technology that, in contrast to RNAi, acts at the post-translational level (i.e., it works directly on the protein level by inducing a specific protein aggregation of a chosen target). Protein aggregation is essentially a misfolding event that occurs through the formation of intermolecular beta-sheets resulting in a functional knockout of a selected target. Through the use of a dedicated algorithm, it is possible to accurately predict which amino acidic stretches in a chosen target protein sequence have the highest self-associating tendency (A. M. Fernandez-Escamilla et al. (2004), Nat. Biotechnol. 22(10):1302-6. By expressing these specific peptides in the cells, the protein of interest can be specifically targeted by inducing its irreversible aggregation and thus its functional knock-out.

In yet another embodiment, the disclosure encompasses still additional methods for reducing or eliminating the activity of one or more KIX8/KIX9 polypeptides. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides and recombinogenic oligonucleotide bases. Such vectors and methods of use are known in the art. See, for example, U.S. Pat. Nos. 5,565,350; 5,731,181; 5,756,325; 5,760,012; 5,795,972 and 5,871,984, each of which are incorporated herein by this reference. Where polynucleotides are used to decrease or inhibit KIX8/KIX9 activity, it is recognized that modifications of the exemplary sequences disclosed herein may be made as long as the sequences act to decrease or inhibit expression of the corresponding mRNA. Thus, for example, polynucleotides having at least 70%, 80%, 85%, 86%, 87%, 88%, 89%, 90%, 91%, 92%, 93%, 94%, 95%, 96%, 97%, 98%, 99%, or 100% sequence identity to the exemplary sequences disclosed herein (e.g., SEQ ID NOS:1, 2, 3 or 4 and orthologous plant sequences exemplified in Examples 9 and 10 may be used. Furthermore, portions or fragments of the exemplary sequences or portions or fragments of polynucleotides sharing a particular percent sequence identity to the exemplary sequences may be used to disrupt the expression of the target gene. Generally, fragments or sequences of at least 10, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 250, 260, 280, 300, 350, 400, 450, 500, 600, 700, 800, 900, 1000, or more contiguous nucleotides, or greater of, for example, SEQ ID NOS:1, 2, 3, 4 or a plant orthologous nucleotide sequence thereof may be used. It is recognized that in particular embodiments, the complementary sequence of such sequences may be used. For example, hairpin constructs comprise both a sense sequence fragment and a complementary, or antisense, sequence fragment corresponding to the gene of interest. Antisense constructs may share less than 100% sequence identity with the gene of interest, and may comprise portions or fragments of the gene of interest, so long as the object of the embodiment is achieved, i.e., as long as expression of the gene of interest is decreased.

The KIX8/KIX9 nucleic acids that may be used for this disclosure comprise a KIX8/KIX9 polynucleotide selected from the group consisting of:
(a) a polynucleotide encoding a KIX8/KIX9 polypeptide and conservatively modified and polymorphic variants thereof, such as SEQ ID NO:1, 2, 3 or 4;
(b) a polynucleotide having at least 35%, 40%, 45%, 50%, 55%, 60%, 65%, 70% sequence identity with polynucleotides of (a);
(c) a fragment of a polynucleotide encoding a KIX8/KIX9 polypeptide; and
(d) complementary sequences of polynucleotides of (a), (b), or (c).

A plant orthologous sequence of KIX8 or KIX9 is herein further abbreviated as a plant orthologous sequence and means a sequence of KIX8 or KIX9 with at least 35%, 40%, 45%, 50%, 55%, 60%, 65% or 70% identity at the nucleotide level with SEQ ID NO:1, 2, 3 or 4.

Thus, in some embodiments, the method comprises introducing at least one polynucleotide sequence comprising a KIX8/KIX9 nucleic acid sequence, or subsequence thereof, into a plant cell, such that the polynucleotide sequences are linked to a plant-expressible promoter in a sense or antisense orientation, and where the polynucleotide sequences comprise, e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, about 99.5% or more sequence identity to SEQ ID NOS:1, 2, 3, 4 or a plant orthologous nucleotide sequence thereof or a subsequence thereof or a complement thereof.

In another embodiment, the disruption is effected by introducing into the plant cell at least one polynucleotide sequence comprising one or more subsequences of a KIX8/KIX9 nucleic acid sequence configured for RNA silencing or interference.

In other embodiments, the methods of the disclosure are practiced with a polynucleotide comprising a member selected from the group consisting of: (a) a polynucleotide or a complement thereof, comprising, e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, about 99.5% or more sequence identity to SEQ ID NOS:1, 2, 3, 4 or a plant orthologous gene sequence thereof or a subsequence thereof, or a conservative variation thereof; (b) a polynucleotide, or a complement thereof, encoding a polypeptide sequence of SEQ ID NOS:1, 2, 3, 4 or a plant orthologous gene sequence thereof or a subsequence thereof, or a conservative variation thereof; (c) a polynucleotide, or a complement thereof, that hybridizes under stringent conditions over substantially the entire length of a polynucleotide subsequence comprising at least 100 contiguous nucleotides of SEQ ID NOS:1, 2, 3, 4 or a plant orthologous gene sequence or that hybridizes to a polynucleotide sequence of (a) or (b); and (d) a polynucleotide that is at least about 85% identical to a polynucleotide sequence of (a), (b) or (c).

In particular embodiments, a heterologous polynucleotide is introduced into a plant, wherein the heterologous polynucleotide is selected from the group consisting of: a) a nucleic acid comprising a KIX8/KIX9 nucleic acid; b) a nucleic acid comprising at least 15 contiguous nucleotides of the complement of a KIX8/KIX9 nucleic acid; and c) a nucleic acid encoding a transcript that is capable of forming a double-stranded RNA (e.g., a hairpin) and mediated RNA interference of a KIX8/KIX9 nucleic acid, wherein the nucleic acid comprises a first nucleotide sequence comprising at least 20 contiguous nucleotides of a KIX8/KIX9 nucleic acid, and a second nucleotide sequence comprising the complement of the first nucleotide sequence.

In other particular embodiments, the methods comprise introducing into a plant a heterologous polynucleotide selected from the group consisting of: a) the nucleotide sequence set forth in SEQ ID NOS:1, 2, 3, 4 or a plant orthologous gene sequence or a complete complement thereof; b) a nucleotide sequence having at least 70%, at least 75%, at least 80%, at least 85%, at least 90%, at least 91%, at least 92%, at least 93%, at least 94%, at least 95%, at least 96%, at least 97%, at least 98%, at least 99% or greater sequence identity to SEQ ID NOS:1, 2, 3, 4 or a plant orthologous sequence thereof, or a complete complement thereof; c) a nucleotide sequence encoding the polypeptide sequence of SEQ ID NOS:1, 2, 3, 4 or a plant orthologous gene sequence thereof; d) a nucleotide sequence encoding a polypeptide sequence having at least 70%, at least 75%, at least 80%, at least 85%, at least 90%, at least 91%, at least 92%, at least 93%, at least 94%, at least 95%, at least 96%, at least 97%, at least 98%, at least 99% or greater sequence identity to SEQ ID NO:2, 4, 6, 8, 10, 12, 14, 16, 18, 20, 22 or 24; e) a nucleotide sequence comprising at least 15 contiguous nucleotides of SEQ ID NOS:1, 2, 3, 4 or a plant orthologous gene sequence thereof; f) a nucleotide sequence comprising at least 15 contiguous nucleotides of the complement of SEQ ID NOS:1, 2, 3, 4 or a plant orthologous gene sequence thereof; and g) a nucleotide sequence encoding a transcript that is capable of forming a double-stranded RNA (e.g., hairpin) and mediating RNA interference of a KIX8/KIX9 nucleic acid, wherein the nucleotide sequence comprises at least 20 contiguous nucleotides of SEQ ID NOS:1, 2, 3, 4 or a plant orthologous gene sequence thereof, and the complement thereof.

In other embodiments, the heterologous polynucleotide comprises at least 500 contiguous nucleotides of SEQ ID NOS:1, 2, 3, 4 or a plant orthologous gene sequence thereof and the complement thereof. In some of these embodiments, the heterologous polynucleotide encodes a transcript that is capable of forming a double-stranded RNA (e.g., hairpin) and mediating RNA interference of a KIX8/KIX9 nucleic acid. In some of these embodiments, the plant comprises an mRNA encoded by a polynucleotide having the target sequence set forth in SEQ ID NOS:1, 2, 3, 4 or a plant orthologous gene sequence thereof.

This disclosure provides methods utilizing, inter alia, isolated nucleic acids of RNA, DNA, homologs, paralogous genes and orthologous genes and/or chimeras thereof, comprising a KIX8/KIX9 polynucleotide. This includes naturally occurring as well as synthetic variants and homologs of the sequences.

The terms “isolated” or “isolated nucleic acid” or “isolated protein” refer to material, such as a nucleic acid or a protein, that is substantially or essentially free from components that normally accompany or interact with it as found in its naturally occurring environment. The isolated material optionally comprises material not found with the material in its natural environment. Preferably, an “isolated” nucleic acid is free of sequences (preferably protein-encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Sequences homologous, i.e., that share significant sequence identity or similarity, to those provided herein derived from maize, Arabidopsis thaliana or from other plants of choice, can also be used in the methods of the disclosure. Homologous sequences can be derived from other dicots and, in particular, agriculturally important plant species including, but not limited to, crops such as soybean, potato, cotton, rape, oilseed rape (including canola), sunflower, alfalfa, clover, or fruits and vegetables, such as banana, blackberry, blueberry, strawberry and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, tomatillo, watermelon, rosaceous fruits (such as apple, peach, pear, cherry and plum) and vegetable brassicas (such as broccoli, cabbage, cauliflower, Brussels sprouts and kohlrabi). Other crops, including fruits and vegetables, whose phenotype can be changed and which comprise homologous sequences include barley; rye; millet; sorghum; currant; avocado; citrus fruits such as oranges, lemons, grapefruit and tangerines, artichoke, cherries; nuts such as the walnut and peanut; endive; leek; roots such as arrowroot, beet, cassava, turnip, radish, yam and sweet potato and beans. The homologous sequences may also be derived from woody species, such pine, poplar and eucalyptus or mint or other labiates.

Homologous sequences as described above can comprise orthologous or paralogous sequences. Several different methods are known by those of skill in the art for identifying and defining these functionally homologous sequences. Three general methods for defining orthologous and paralogous genes are described; an ortholog, paralog or homolog may be identified by one or more of the methods described below. Orthologs and paralogs are evolutionarily related genes that have similar sequence and similar functions. Orthologs are structurally related genes in different species that are derived by a speciation event. Paralogs are structurally related genes within a single species that are derived by a duplication event. Within a single plant species, gene duplication may result in two copies of a particular gene, giving rise to two or more genes with similar sequence and often similar function known as paralogs. A paralog is, therefore, a similar gene formed by duplication within the same species. Paralogs typically cluster together or in the same clade (a group of similar genes) when a gene family phylogeny is analyzed using programs such as CLUSTAL (Thompson, et al. (1994) Nucleic Acid Res. 22:4673-4680; Higgins, et al. (1996) Methods Enzymol. 266:383-402). Groups of similar genes can also be identified with pair-wise BLAST analysis (Feng and Doolittle (1987) J. Mol. Evol. 25:351-360). Orthologous sequences can also be identified by a reciprocal BLAST strategy. Once an orthologous sequence has been identified, the function of the ortholog can be deduced from the identified function of the reference sequence. Orthologous genes from different organisms have highly conserved functions, and very often essentially identical functions (Lee, et al. (2002) Genome Res. 12:493-502; Remm, et al. (2001) J. Mol. Biol. 314:1041-1052). Paralogous genes, which have diverged through gene duplication, may retain similar functions of the encoded proteins. In such cases, paralogs can be used interchangeably with respect to certain embodiments of this disclosure (for example, transgenic expression of a coding sequence).

KIX8/KIX9 polynucleotides, such as those disclosed herein, can be used to isolate homologs, paralogs and orthologs. In this manner, methods such as PCR, hybridization, and the like can be used to identify such sequences based on their sequence homology to the KIX8/KIX9 polynucleotide. In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially mismatched primers, and the like.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence-based amplification (NASBA, Cangene, Mississauga, Ontario), Q-Beta Replicase systems, transcription-based amplification system (TAS) and strand displacement amplification (SDA). See, e.g., Diagnostic Molecular Microbiology: Principles and Applications, Persing, et al., eds., American Society for Microbiology, Washington, D.C. (1993). The product of amplification is termed an amplicon.

In hybridization techniques, all or part of a known polynucleotide is used as a probe that selectively hybridizes to other nucleic acids comprising corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as P, or any other detectable marker. Thus, for example, probes for hybridization can be made by labeling synthetic oligonucleotides based on the KIX8/KIX9 sequences disclosed herein. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire KIX8/KIX9 sequences disclosed herein, or one or more portions thereof, may be used as probes capable of specifically hybridizing to corresponding KIX8/KIX9 sequences and messenger RNAs. To achieve specific hybridization under a variety of conditions, such probes include sequences that are unique among KIX8/KIX9 sequences and are at least about 10, 12, 15, 16, 17, 18, 19, 20, 21, 22, 23, 24, 25, 30, 35, 40, 50, 60, 70, 80, 90, or more nucleotides in length. Such probes may be used to amplify corresponding KIX8/KIX9 sequences from a chosen plant by PCR. This technique may be used to isolate additional coding sequences from a desired plant or as a diagnostic assay to determine the presence of coding sequences in a plant. Hybridization techniques include hybridization screening of plated nucleic acid (e.g., DNA) libraries (either plaques or colonies; see, for example, Sambrook et al. (1989) Molecular Cloning: A Laboratory Manual (2d ed., Cold Spring Harbor Laboratory Press, Plainview, N.Y.). By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, (1987) Guide To Molecular Cloning Techniques, from the series Methods in Enzymology, vol. 152, Academic Press, Inc., San Diego, Calif.; Sambrook, et al., (1989) Molecular Cloning: A Laboratory Manual, 2^{nd} ed., vols. 1-3; and Current Protocols in Molecular Biology, Ausubel, et al., eds, Current Protocols, a joint venture between Greene Publishing Associates, Inc. and John Wiley & Sons, Inc. (1994 Supplement).

Hybridization of such sequences may be carried out under stringent conditions. The terms “stringent conditions” or “stringent hybridization conditions” include reference to conditions under which a probe will hybridize to its target sequence, to a detectably greater degree than other sequences (e.g., at least two-fold over background). Stringent conditions are sequence-dependent and will be different in different circumstances. By controlling the stringency of the hybridization and/or washing conditions, target sequences can be identified, which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS at 37° C., and a wash in 1× to 2×SSC at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Specificity is typically the function of post-hybridization washes, the critical factors being the ionic strength and temperature of the final wash solution.

For DNA-DNA hybrids, the Tₘ can be approximated from the equation of Meinkoth and Wahl, (1984) Anal. Biochem. 138:267-84: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier, New York (1993); and Current Protocols in Molecular Biology, chapter 2, Ausubel, et al., eds, Greene Publishing and Wiley-Interscience, New York (1995). Unless otherwise stated in this disclosure, high stringency is defined as hybridization in 4×SSC, 5×Denhardt's (5 g Ficoll, 5 g polyvinylpyrrolidone, 5 g bovine serum albumin in 500 ml of water), 0.1 mg/ml boiled salmon sperm DNA and 25 mM Na phosphate at 65° C. and a wash in 0.1×SSC, 0.1% SDS at 65° C.

The term “selectively hybridizes” includes reference to hybridization, under stringent hybridization conditions, of a nucleic acid sequence to a specified nucleic acid target sequence to a detectably greater degree (e.g., at least two-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other. The term “hybridization complex” includes reference to a duplex nucleic acid structure formed by two single-stranded nucleic acid sequences selectively hybridized with each other.

In this disclosure, a “plant-expressible promoter” comprises regulatory elements, which mediate the expression of a coding sequence segment in plant cells. For expression in plants, the nucleic acid molecule must be linked operably to or comprise a suitable promoter that expresses the gene at the right point in time and with the required spatial expression pattern. For the identification of functionally equivalent promoters, the promoter strength and/or expression pattern of a candidate promoter may be analyzed, for example, by operably linking the promoter to a reporter gene and assaying the expression level and pattern of the reporter gene in various tissues of the plant. Suitable well-known reporter genes include, for example, beta-glucuronidase or beta-galactosidase. The promoter activity is assayed by measuring the enzymatic activity of the beta-glucuronidase or beta-galactosidase.

The promoter strength and/or expression pattern may then be compared to that of a reference promoter (such as the one used in the methods of this disclosure). Alternatively, promoter strength may be assayed by quantifying mRNA levels or by comparing mRNA levels of the nucleic acid used in the methods of this disclosure, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6:986-994). Generally, by “weak promoter” is intended a promoter that drives expression of a coding sequence at a low level. By “low level” is intended at levels of about 1/10,000 transcripts to about 1/100,000 transcripts, to about 1/500,000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter, in particular, at a level that is in all instances below that obtained when under the control of a 35S CaMV promoter.

The term “operably linked” as used herein refers to a functional linkage between the promoter sequence and the gene of interest, such that the promoter sequence is able to initiate transcription of the gene of interest.

A “constitutive promoter” refers to a promoter that is transcriptionally active during most, but not necessarily all, phases of growth and development and under most environmental conditions, in at least one cell, tissue or organ. An “ubiquitous” promoter is active in substantially all tissues or cells of an organism. A developmentally regulated promoter is active during certain developmental stages or in parts of the plant that undergo developmental changes. An inducible promoter has induced or increased transcription initiation in response to a chemical (for a review see Gatz 1997, Ann. Rev. Plant Physiol. Plant Mol. Biol., 48:89-108), environmental or physical stimulus, or may be “stress-inducible,” i.e., activated when a plant is exposed to various stress conditions, or a “pathogen-inducible,” i.e., activated when a plant is exposed to various pathogens. An organ-specific or tissue-specific promoter is one that is capable of preferentially initiating transcription in certain organs or tissues, such as the leaves, roots, seed tissue, etc. For example, a “root-specific promoter” is a promoter that is transcriptionally active predominantly in plant roots, substantially to the exclusion of any other parts of a plant, while still allowing for any leaky expression in these other plant parts. Promoters able to initiate transcription in certain cells only are referred to herein as “cell-specific.” A seed-specific promoter is transcriptionally active predominantly in seed tissue, but not necessarily exclusively in seed tissue (in cases of leaky expression). The seed-specific promoter may be active during seed development and/or during germination. The seed-specific promoter may be endosperm/aleurone/embryo specific. Examples of seed-specific promoters are given in Qing Qu and Takaiwa (Plant Biotechnol. J. 2:113-125, 2004), which disclosure is incorporated by reference herein as if fully set forth. A “green tissue-specific promoter” as defined herein is a promoter that is transcriptionally active predominantly in green tissue, substantially to the exclusion of any other parts of a plant, while still allowing for any leaky expression in these other plant parts.

Examples of constitutive promoters capable of driving such expression are the 35S and eIF-4A promoters.

The term “terminator” encompasses a control sequence that is a DNA sequence at the end of a transcriptional unit that signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

“Selectable or screenable marker,” “selectable or screenable marker gene” or “reporter gene” includes any gene that confers a phenotype on a cell in which it is expressed to facilitate the identification and/or selection of cells that are transfected or transformed with a nucleic acid construct of the disclosure. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptll that phosphorylates neomycin and kanamycin, or hpt, phosphorylating hygromycin, or genes conferring resistance to, for example, bleomycin, streptomycin, tetracycline, chloramphenicol, ampicillin, gentamycin, geneticin (G418), spectinomycin or blasticidin), to herbicides (for example, bar that provides resistance to BASTA®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilization of xylose, or anti-nutritive markers such as the resistance to 2-deoxyglucose). Expression of visual marker genes results in the formation of color (for example, β-glucuronidase, GUS or β-galactosidase with its colored substrates, for example X-Gal), luminescence (such as the luciferin/luciferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can, for example, be used in mutants in which these genes are not functional by, for example, deletion by conventional methods. Furthermore, nucleic acid molecules encoding a selectable marker can be introduced into a host cell on the same vector that comprises the sequence encoding the polypeptides of the disclosure or used in the methods of the disclosure, or else in a separate vector. Cells that have been stably transfected with the introduced nucleic acid can be identified, for example, by selection (for example, cells that have integrated the selectable marker survive, whereas the other cells die).

Since the marker genes, particularly genes for resistance to antibiotics and herbicides, are no longer required or are undesired in the transgenic host cell once the nucleic acids have been successfully introduced, the process according to the disclosure for introducing the nucleic acids advantageously employs techniques that enable the removal or excision of these marker genes. One such method is what is known as “co-transformation.” The co-transformation method employs two vectors simultaneously for the transformation, one vector bearing the nucleic acid according to the disclosure and a second bearing the marker gene(s). A large proportion of transformants receives or, in the case of plants, comprises (up to 40% or more of the transformants) both vectors.

In case of transformation with Agrobacteria, the transformants usually receive only a part of the vector, i.e., the sequence flanked by the T-DNA, which usually represents the expression cassette. The marker genes can be subsequently removed from the transformed plant by performing crosses. In another method, marker genes integrated into a transposon are used for the transformation, together with desired nucleic acid (known as the Ac/Ds technology). The transformants can be crossed with a transposase source or the transformants are transformed with a nucleic acid construct conferring expression of a transposase, transiently or stable. In some cases (approximately 10%), the transposon jumps out of the genome of the host cell once transformation has successfully taken place and is lost. In a further number of cases, the transposon jumps to a different location. In these cases, the marker gene must be eliminated by performing crosses.

In microbiology, techniques were developed that make possible, or facilitate, the detection of such events. A further advantageous method relies on what is known as recombination systems, whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the disclosure is possible. Similarly, marker genes can be excised using one or more rare-cleaving double-strand break-inducing enzymes such as meganucleases (naturally occurring or engineered to recognize a specific DNA sequence), zinc finger nucleases, TALE nucleases and the like, if recognition sites for such enzymes are present in the vicinity of the marker gene. Excision can occur via homologous recombination if homology regions flank the marker gene, or via non-homologous end-joining with two recognition sites flanking the marker gene.

For the purposes of the disclosure, “transgenic,” “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the disclosure. The term “nucleic acid molecule” as used interchangeably with the term “polynucleotide” in accordance with this disclosure, includes DNA, such as cDNA or genomic DNA, and RNA.

A transgenic plant for the purposes of the disclosure is thus understood as meaning, as above, that the nucleic acids used in the method of the disclosure (e.g., the chimeric genes) are not present in, or originating from, the genome of the plant, or are present in the genome of the plant but not at their natural locus in the genome of the plant, it being possible for the nucleic acids to be expressed homologously or heterologously. However, as mentioned, transgenic also means that, while the nucleic acids according to the disclosure or used in the disclosed method are at their natural position in the genome of a plant, the sequence has been modified with regard to the natural sequence, and/or that the regulatory sequences of the natural sequences have been modified. Transgenic is preferably understood as meaning the expression of the nucleic acids according to the disclosure at an unnatural locus in the genome, i.e., homologous or, heterologous expression of the nucleic acids takes place. Preferred transgenic plants are mentioned herein.

The term “expression” or “gene expression” means the transcription of a specific gene or specific genes or specific genetic construct. The term “expression” or “gene expression,” in particular, means the transcription of a gene or genes or genetic construct into structural RNA (rRNA, tRNA) or mRNA with or without subsequent translation of the latter into a protein. The process includes transcription of DNA and processing of the resulting mRNA product.

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of this disclosure and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, mega-gametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (F. A. Krens, et al. (1982) Nature 296:72-74; I. Negrutiu et al. (1987) Plant Mol. Biol. 8:363-373); electroporation of protoplasts (R. D. Shillito et al. (1985) Bio./Technol. 3:1099-1102); microinjection into plant material (A. Crossway et al. (1986) Mol. Gen. Genet. 202:179-185); DNA or RNA-coated particle bombardment (T. M. Klein et al. (1987) Nature 327:70) infection with (non-integrative) viruses and the like. Transgenic plants, including transgenic crop plants, are preferably produced via Agrobacterium-mediated transformation.

An advantageous transformation method is the transformation in planta. To this end, it is possible, for example, to allow the agrobacteria to act on plant seeds or to inoculate the plant meristem with agrobacteria. It has proved particularly expedient in accordance with the disclosure to allow a suspension of transformed agrobacteria to act on the intact plant or at least on the flower primordia. The plant is subsequently grown on until the seeds of the treated plant are obtained (Clough and Bent, Plant J. (1998) 16:735-743). Methods for Agrobacterium-mediated transformation of rice include well-known methods for rice transformation, such as those described in any of the following: European patent application EP1198985, Aldemita and Hodges (Planta 199:612-617, 1996); Chan et al. (Plant Mol. Biol. 22(3):491-506, 1993), Hiei et al. (Plant J. 6(2):271-282, 1994), the disclosures of which are incorporated herein by this reference as if fully set forth. In the case of corn transformation, the preferred method is as described in either Ishida et al. (Nat. Biotech. 14(6):745-750, 1996) or Frame et al. (Plant Physiol. 129(1):13-22, 2002), which disclosures are incorporated by reference herein as if fully set forth. The methods are further described by way of example in B. Jenes et al., Techniques for Gene Transfer in Transgenic Plants, Vol. 1, Engineering and Utilization, eds. S. D. Kung and R. Wu, Academic Press (1993) 128-143; and in Potrykus, Annu. Rev. Plant Physiol. Plant Mol. Biol. 42 (1991) 205-225). The nucleic acids or the construct to be expressed is preferably cloned into a vector, which is suitable for transforming Agrobacterium tumefaciens, for example, pBin19 (Bevan et al. (1984) Nucl. Acids Res. 12-8711). Agrobacteria transformed by such a vector can then be used in known manner for the transformation of plants, such as plants used as a model, like Arabidopsis or crop plants such as, by way of example, tobacco plants, for example, by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877, or is known inter alia from F. F. White, Vectors for Gene Transfer in Higher Plants in Transgenic Plants Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and, in particular, those cells that develop into gametes. In this case, the transformed gametes follow the natural plant development, giving rise to transgenic plants. Thus, for example, seeds of Arabidopsis are treated with agrobacteria and seeds are obtained from the developing plants, of which a certain proportion is transformed and thus transgenic (K. A. Feldman and M. D. Marks (1987), Mol. Gen. Genet. 208:1-9; K. Feldmann (1992) in C. Koncz, N-H Chua and J. Shell, eds, Methods in Arabidopsis Research, Word Scientific, Singapore, pp. 274-289).

Alternative methods are based on the repeated removal of the inflorescences and incubation of the excision site in the center of the rosette with transformed agrobacteria, whereby transformed seeds can likewise be obtained at a later point in time (Chang (1994) Plant J. 5:551-558; Katavic (1994) Mol. Gen. Genet. 245:363-370). However, an especially effective method is the vacuum infiltration method with its modifications such as the “floral dip” method. In the case of vacuum infiltration of Arabidopsis, intact plants under reduced pressure are treated with an agrobacterial suspension (N. Bechthold (1993) C.R. Acad. Sci. Paris Life Sci. 316:1194-1199), while in the case of the “floral dip” method, the developing floral tissue is incubated briefly with a surfactant-treated agrobacterial suspension (S. J. Clough and A. F. Bent (1998) The Plant J. 16:735-743). A certain proportion of transgenic seeds are harvested in both cases, and these seeds can be distinguished from non-transgenic seeds by growing under the above-described selective conditions.

In addition, the stable transformation of plastids is advantageous because plastids are inherited maternally is most crops, reducing or eliminating the risk of transgene flow through pollen. The transformation of the chloroplast genome is generally achieved by a process that has been schematically displayed in Klaus et al., 2004 (Nature Biotechnology 22(2):225-229). Briefly, the sequences to be transformed are cloned together with a selectable marker gene between flanking sequences homologous to the chloroplast genome. These homologous flanking sequences direct site-specific integration into the plastome. Plastidal transformation has been described for many different plant species and an overview is given in Bock (2001) Transgenic plastids in basic research and plant biotechnology, J. Mol. Biol. 2001 Sep. 21; 312 (3):425-38; or P. Maliga (2003) Progress towards commercialization of plastid transformation technology, Trends Biotechnol. 21:20-28. Further biotechnological progress has recently been reported in the form of marker-free plastid transformants, which can be produced by a transient co-integrated maker gene (Klaus et al., 2004, Nature Biotechnology 22(2), 225-229).

The genetically modified plant cells can be regenerated via all methods with which the skilled worker is familiar. Suitable methods can be found in the above-mentioned publications by S. D. Kung and R. Wu, Potrykus or Hofgen and Willmitzer.

Generally after transformation, plant cells or cell groupings are selected for the presence of one or more markers, which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance, using Southern analysis, for the presence of the gene of interest, copy number and/or genomic organization. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells, clonal transformants (e.g., all cells transformed to contain the expression cassette), grafts of transformed and untransformed tissues (e.g., in plants, a transformed rootstock grafted to an untransformed scion).

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

In some embodiments, the plant cell according to the disclosure is non-propagating or cannot be regenerated into a plant.

Plants that are particularly useful in the methods of the disclosure include dicotyledonous plants including fodder or forage legumes, ornamental plants, food crops, trees or shrubs.

In a particularly preferred embodiment, the plants comprise a Glycine max and Gossypium species. In other embodiments, the term “plant” encompasses species, hybrids and varieties of trees such as Salix, Populus, and Eucalyptus genera.

In view of the above, it should be understood that the “plant biomass” for use in methods requiring or exploiting plant cell wall carbohydrates, for example, biofuel production, may comprise material or matter derived from modified forms (i.e., forms exhibiting modulated expression of one or more KIX8/KIX9s of any of the plants described herein. Further, a skilled person will appreciate that the term “biomass” may comprise any part of a plant including, for example, the stem, flower (including seed heads, etc.), root and leaves. Where a modified plant provided by this disclosure exhibits modified lignin content throughout its cells and tissues, any part of that plant may yield biomass that is useful as feedstock for methods requiring plant carbohydrate extraction or methods of producing biofuel; of particular use are the stems and roots.

The choice of suitable control plants is a routine part of an experimental setup and may include corresponding wild-type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant” as used herein refers not only to whole plants, but also to plant parts, including seeds and seed parts.

All publications and patent applications in this specification are indicative of the level of ordinary skill in the art to which this disclosure pertains. All publications and patent applications are herein incorporated by reference to the same extent as if each individual publication or patent application was specifically and individually indicated by reference.

Many modifications and other embodiments of the disclosure set forth herein will come to mind to one skilled in the art to which this disclosure pertains, having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### Examples and Materials and Methods

### 1. Down-Regulation of PPDs Gene Expression in Col-0 Background Increases Leaf Area and Cell Number

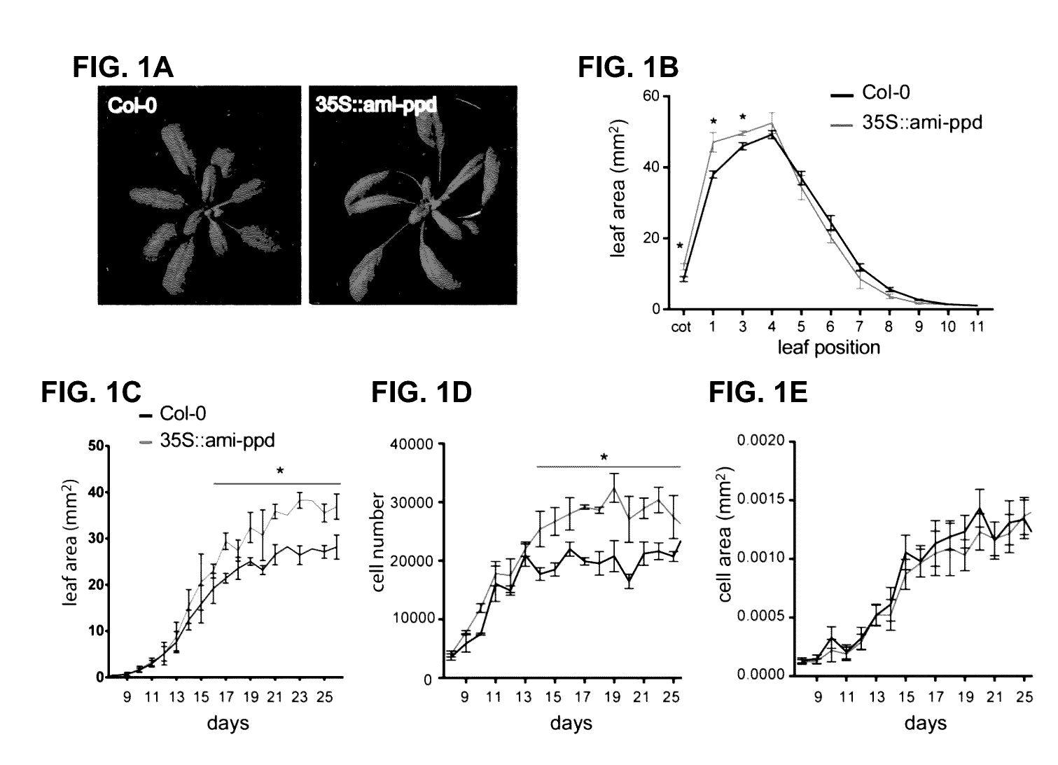

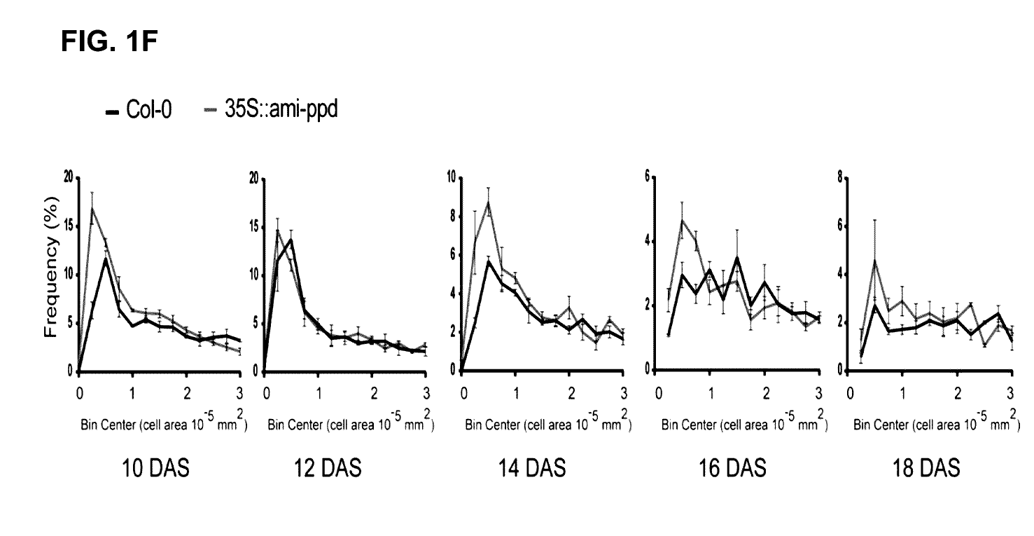

In an Arabidopsis thaliana Ler background, it was published that the deletion of the PPD genes (Δppd) leads to a change in leaf shape and an increase of cotyledon and leaf area (see WO2007/105967). A transgenic line (ami-ppd) was generated over-expressing an artificial micro-RNA targeting PPD1 and PPD2 in the Arabidopsis Col-0 background and confirmed that also, in this genetic background, plants form dome-shaped leaves (see FIG. 1A). In order to analyze the leaf growth phenotype in this transgenic, leaf series were done from plants grown in vitro for 21 DAS to measure the area of individual leaves. Leaf area is increased when the expression of the PPD genes is reduced and found that cotyledons, the first pair of leaves and leaf 3 are larger compared to wild-type (see FIG. 1B). It was shown before in the art (see WO2007/105967) that the increased leaf area in the Δppd mutant results from a prolonged division of dispersed meristematic cells (or meristemoid) in the epidermis. In order to identify when, during leaf development, these cellular changes trigger the formation of larger leaves in the ami-ppd plants compared to wild-type, leaves 1 and 2 from these two genotypes were harvested daily for area measurement and quantification of cell number and area from the epidermis. At early time points, the leaves of the ami-ppd lines and wild-type are similar in size, but after 13 days, leaf area becomes larger in the mutants and this difference becomes significant from 16 DAS (see FIG. 1C). At maturity (25 DAS), the average cell area is not different from WT (see FIG. 1E), therefore, showing that the increase in leaf area is due to an increase in cell number (see FIG. 1D). This increase in cell number, observed early during development, becomes significant after 14 DAS. Although average cell area is similar in ami-ppd and wild-type, the analysis of cell size distribution from 10 DAS to 18 DAS showed that in ami-ppd leaves, the proportion of small cells (between 2.5-7.5 10⁻⁵ mm²) is increased compared to wild-type from 10 DAS (see FIG. 1F). The data herein show that in Col-0 background, down-regulation of PPD gens leads to an increase in leaf area resulting from an increase in cell number observed early during development.

### 2. Genes Differentially Expressed Upon PPD Down-Regulation

To obtain more insight into the molecular changes associated with the down-regulation of PPD, RNA was extracted from the first leaf pairs of ami-ppd and WT plants at 13 DAS, time point at which differences in leaf area start to be visible (see FIG. 1C), and subjected to micro-array transcript profiling. It was found that 49 genes (excluding PPD2) were differentially expressed (P value<0.05) in the ami-ppd line compared to WT, with 36 up-regulated and 13 down-regulated genes (see Tables 1 and 2). Differential transcripts were investigated with Page-Man (Usadel et al., 2006) and the classification Super Viewer tool (Provart et al., 2003) to calculate the functional over-representation of MapMan categories and GO categories, respectively. Due to the low number of genes differentially expressed, only one over-represented category was fiynd, “hormone metabolism,” and, more particularly, “auxin-regulated” genes: two members of the auxin-responsive GH3 family DFL1/GH3.6 and GH3.3-encoding indole-3-acetic acid amido synthetases, and two members of the SAUR-like family (AT2G37030 and AT4G00880).

Because PPD have been described to be involved in the regulation of the stomatal meristemoid division (White, 2006), the dataset was compared to a list of known stomatal development and patterning genes (Pilliteri and Dong, 2013) and to publicly available data sets corresponding to molecular profiling of stomatal meristemoids (Pilliteri et al., 2011). Among the genes involved in stomatal development (Pilliteri and Dong, 2013), a significant increase in expression of SPCH (P.value<0.05), Table 1) was found. In addition, although their difference in expression was not significant, it was observed that another gene involved in the differentiation of stomata (MUTE), five genes involved in spacing and patterning (ERL2, TMM, SDD1, EPF1 and EPF2) and the two known genes involved in polarity and division asymmetry (BASL and POLAR) are also up-regulated in the leaves of ami-ppd compared to wild-type. The expression of these genes was analyzed by quantitative RT-PCR in a time course experiment in which the first leaf pairs from ami-ppd and wild-type plants were harvested from 11 to 16 DAS and used for RNA extraction. For most of these genes, the increased expression in the ami-ppd line at 13 DAS was confirmed. It was also observed that the expression SPCH, MUTE and TMM was already higher at 11 DAS and stayed higher until 13 DAS for SPCH and MUTE and 14 DAS for TMM. Although less pronounced, the other genes also showed an increased expression in the line over-expressing the ami-RNA targeting PPD. It was also found that four genes (CYCD3;2, SPCH, ATSBT1.3 and AT4G29020) up-regulated in the meristemoid-enriched background, scrm-D;mute, are also up-regulated in ami-ppd leaves (see Table 1).

Due to the potential role of PPD in the regulation of cell division, the differentially expressed genes in ami-ppd leaves were also compared to dataset of proliferation-specific genes (Beemster et al., 2005). A second CYCD3, CYCD3;3, was found to be over-expressed in the ami-ppd as well as the gene AT5G43020, encoding a leucine-rich repeat transmembrane protein kinase, which is also specifically expressed in proliferating tissues. It was confirmed, by qRT-PCR, the higher expression of the two CYCD3s in the ami-ppd leaves at 13 DAS but also until 16 DAS. In order to gain further insight into the 49 differentially expressed genes in the ami-ppd line, the online tool CORNET was used to perform co-expression analysis with predefined sets of microarray expression data. These datasets can be divided in several sub-groups such as microarray experiments in which leaf tissues are sampled (leaf), hormone treatment series (hormone), microarray experiments oriented toward growth, development and cell cycle studies (compendium 1) or microarray experiments for which very similar experiments were removed (compendium2). By using these sub-groups of datasets for the co-expression analysis (pearson correlation>0.7), four networks were obtained containing 34 genes of the 49 differentially expressed with 67 connecting edges. One network contains 23 genes connected with 56 edges while a smaller one contains 7 genes connected with 8 edges. Interestingly, among the genes of the large network, the two CYCD3s, SPCH, ATSBT1.3 and AT4G29020 were observed. In this large network, two genes, HMGA and ATSBT1.3, are highly connected with others, with 14 and 10 edges, respectively. This high co-expression of HMGA with other genes comes mainly from leaf dataset experiments. The small network is more related to hormone experiment. In conclusion, few genes were found that differentially expressed in the first pair of leaves of the ami-ppd line at 13 DAS, which are mainly up-regulated. These genes seem, however, to be highly connected in terms of co-expression and they are related to cell division, meristemoid cells and stomatal development.

### 3. Genome-Wide Identification of PPD2 DNA Binding Sites

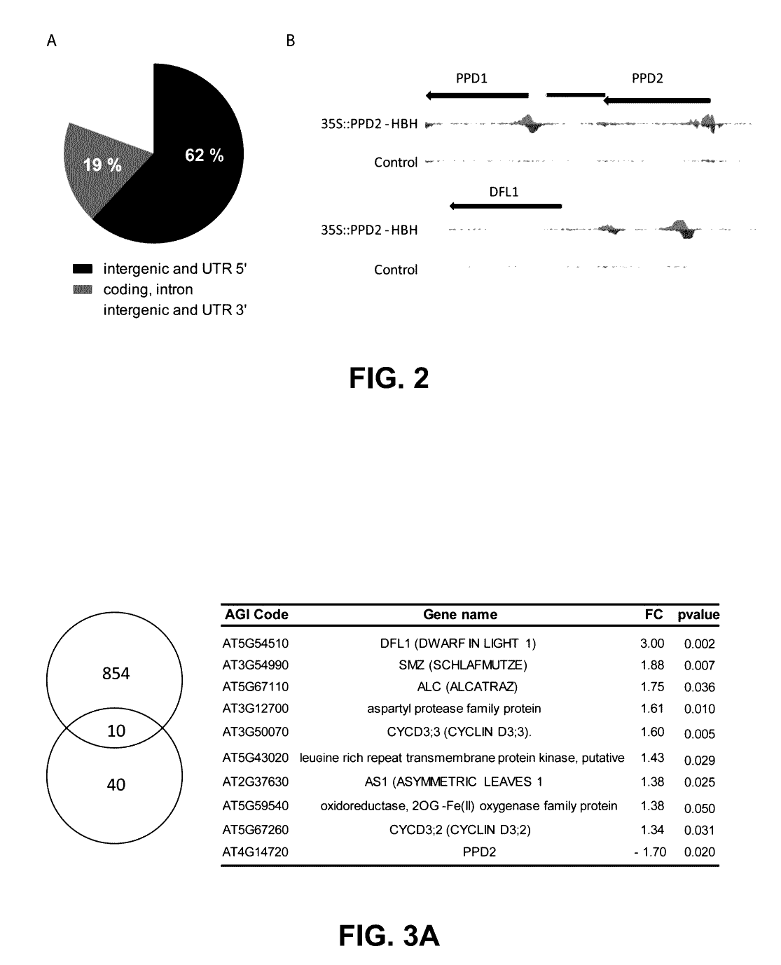

PPD2 encodes a putative transcription factor belonging to the TIFY protein family, a plant-specific group of proteins with a broad range of functions (Zhang et al., 2012). To identify direct target genes of PPD2, tandem chromatin affinity purification (TChAP, ref), a variant of chromatin immunoprecipitation (ChIP), followed by sequencing (TChAP-seq) was performed. Arabidopsis cell suspension cultures over-expressing an HBH-tagged PPD2 were used for the purification of the chromatin bound by PPD2. After sequencing of the purified DNA, a total of 19.61 and 16.57 million reads were obtained for the PPD2-HBH sample and the wild-type control sample (35S::HBH), respectively. 914 peaks representing non-redundant reads and reads mapping uniquely to the genome, and corresponding to 904 genes, were identified to be specific to the PPD2-HBH sample. The analysis of the location of these peak sequences, having an average length of approximately 1700 bp, showed that 81% are situated in the intergenic/UTR regions (62 and 19% in the 5′ and 3′ intergenic/UTR region, respectively), and only 19% in the coding and intron regions (FIG. 2, Panel A). In addition, around 40% of the peaks have their peak summit located between −300 and 100 bp from the translation start site with a maximum between −100 and 0 bp. Among the 914 peak sequences identified, a search for specific motifs was performed by using the RSAT peak-motifs tool (Thomas-Chollier et al., 2012) and found that the motif GmCACGTGkC, containing the G-box sequence (CACGTG) was highly represented. This motif, preferentially located near the peak summit, is present in 506 peak sequences.

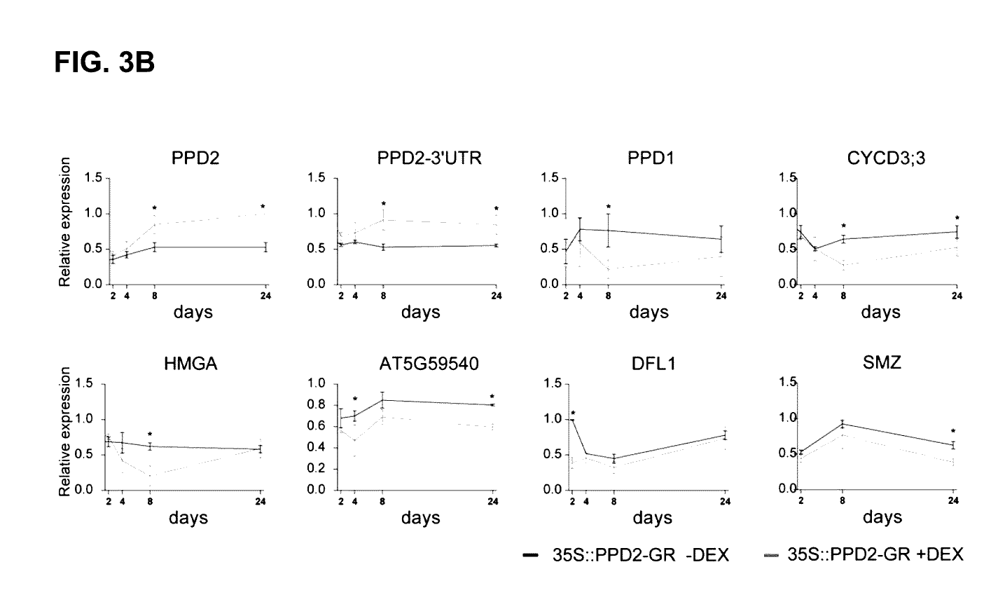

To gain insight in the 904 genes identified after sequencing of the chromatin bound by PPD, Page-Man (Usadel et al., 2006) was used to calculate the functional over-representation of MapMan categories. Four categories were over-represented in this list of genes: “regulation of transcription,” “hormone metabolism,” “protein degradation” and, more particularly, “ubiquitin E3.RING proteins” and UDP glucosyl and glucuronyl transferases.” More than one hundred transcription factor sequences were bound by PPD2, including PPD1 and PPD2 promoters (see FIG. 2, Panel B). These transcription factors belong to different families, including WRKY, homeobox or APETALA2/ethylene responsive element binding protein transcription factors. In the category “hormone metabolism,” genes related to various hormones were found such as DFL1, presenting two binding sites for PPD2 (FIG. 2, Panel B) and involved in auxin metabolism, several ERFs (ERF2; 5, 13) involved in ethylene signal transduction or JAZ3, a TIFY protein involved in jasmonate signaling. The PPD2-HBH TCHAP-seq dataset was compared to the genes differentially expressed in the ami-ppd line. Out of the 49 genes differentially expressed in this line, nine over-expressed genes and PPD2 are found in the list of sequences bound by PPD2 (see FIGS. 3A and 3B). Interestingly, CYCD3;2 and CYCD3;3 are part of these direct targets of PPD2. As for these CYCD3s, the expression of the direct target genes was analyzed over time (11 to 16 DAS) in the first leaf pairs of ami-ppd and wild-type plants by quantitative RT-PCR. The increase in expression of SMZ, DFL1, ALC was confirmed at 13 DAS and already visible at 11 DAS for SMZ, DFL1. Because HMGA and ATSBT1.3, two genes up-regulated in the ami-ppd line, were found to be highly connected in the co-expression network built with CORNET, the presence of a peak in the PPD2-HBH TCHAP dataset was searched in the neighborhood of these two genes by using Genome viewer. For HMGA but not for ATSBT1.3, a peak was found in the 5′ intergenic region showing that PPD2 also binds to the promoter of this gene. The expression over time of HMGA was also higher compared to wild-type from 11 DAS until 16 DAS. In conclusion, the DNA binding sites of the PPD2 proteins were identified by TCHAP-seq and among which eleven genes are up-regulated in the leaves of the ami-ppd line.

### 4. PPD2 Positively or Negatively Regulates the Expression of its Target Genes

The fact that the target genes of PPD2 identified by TCHAP-seq are up-regulated in the ami-ppd line, suggests that PPD2 acts as negative regulator for these targets. In order to verify this hypothesis, homozygous plants containing an inducible gain-of-function construct 35S:PPD2-GR (PPD2-GR) were generated. Nine-day-old PPD2-GR plants grown on MS medium were transferred to medium either containing or not containing dexamethasone (DEX), a glucocorticoid hormone allowing the translocation of PEAPOD2 fused to the rat glucocorticoid receptor (GR) domain to the nucleus. RNA was extracted from the first leaf pairs harvested 2, 4, 8 and 24 hours after transfer. The expression of PPD1, PPD2 was quantified by qRT-PCR and of the genes identified as direct targets of PPD2 by TCHAP-seq, genes were found to be differentially expressed in the ami-ppd line. Upon DEX treatment, PPD2 expression increased over time after 8 hours (see FIG. 3B), which could be due to progressive accumulation of the RNA from the PPD2-GR transgene and/or the activation of endogenous PPD2 expression as PPD2 binds to its own promoter. To verify this, primers located in the 3′-UTR of the PPD2 gene, therefore, only amplifying the endogenous PPD2 sequence were used for qRT-PCR. The expression of the endogenous PPD2 was increased in PPD2-GR transferred to DEX containing medium after 8 hours at a similar level than when primers amplifying both PPD2 sequences are used suggesting that PPD2 is able to activate its own expression. On the contrary, the level of PPD1 expression upon activation of PPD2 expression decreases over time from 4 hours after transfer. Similarly, the expression of CYCD3;3, HMGA and AT5G59540 is decreased after 4 or 8 hours. In the case of DFL and SMZ, a decreased expression was found at only one time point, at 2 hours and 24 hours after PPD2 activation. Remarkably, the expression of CYCD3;2 was up-regulated after 24 hours. In conclusion, PPD2 by binding to its own promoter activates its expression and inhibits the expression of most of the target genes tested such as CYCD3;3 and HMGA.

### 5. PPD2 Protein Contains a Functional ZIM Domain

PPD proteins belong to the class II of the TIFY protein family together with 12 JAZ proteins (ref). In the TIFY proteins, diverse domains are associated with different protein-protein interaction abilities, previously being able to isolate, by using tandem affinity purification (TAP), protein complexes from Arabidopsis cell cultures over-expressing JAZ proteins and to identify their interacting proteins (Pauwels et al., 2010). In order to determine the protein complex associated to PPD2, an updated and more sensitive protocol was applied using Orbitrap mass spectrometry (Eloy et al., 2012). PPD2 was found to interact with the TIFY proteins JAZ3, JAZ12 and TIFY8, suggesting that heterodimerization within the TIFY family is not restricted to the JAZ proteins (see Table 3). To test this hypothesis, all 12 JAZ proteins, PPD1, PPD2 and TIFY8 for interaction with the PPD proteins were tested using yeast two-hybrid (Y2H) assays. These confirmed direct interaction with JAZ3 and TIFY8 and provided evidence of homo- and heterodimerization between PPD1 and PPD2. Moreover, the TAP analysis identified the TPL-adaptor protein NINJA and two proteins of yet unknown function, encoded by At3g24150 and At4g32295 and named KIX8 and KIX9 (see Table 3, Thakur et al., 2013).

Dimerization of JAZ proteins requires the ZIM domain, which is also present in PPD proteins. Therefore, truncated versions of the PPD2 protein were designed comprising different combinations of its N-terminal PPD, central ZIM and C-terminal Jas-like domains and tested these fragments for interaction with JAZ3 in Y2H. These results show that the ZIM domain is necessary and sufficient for interaction with JAZ3 and demonstrate that the PPD2 ZIM domain is a functional protein-protein interaction domain.

6. KIX Proteins Directly Interact with the PPD Domain and are TPL Adaptors

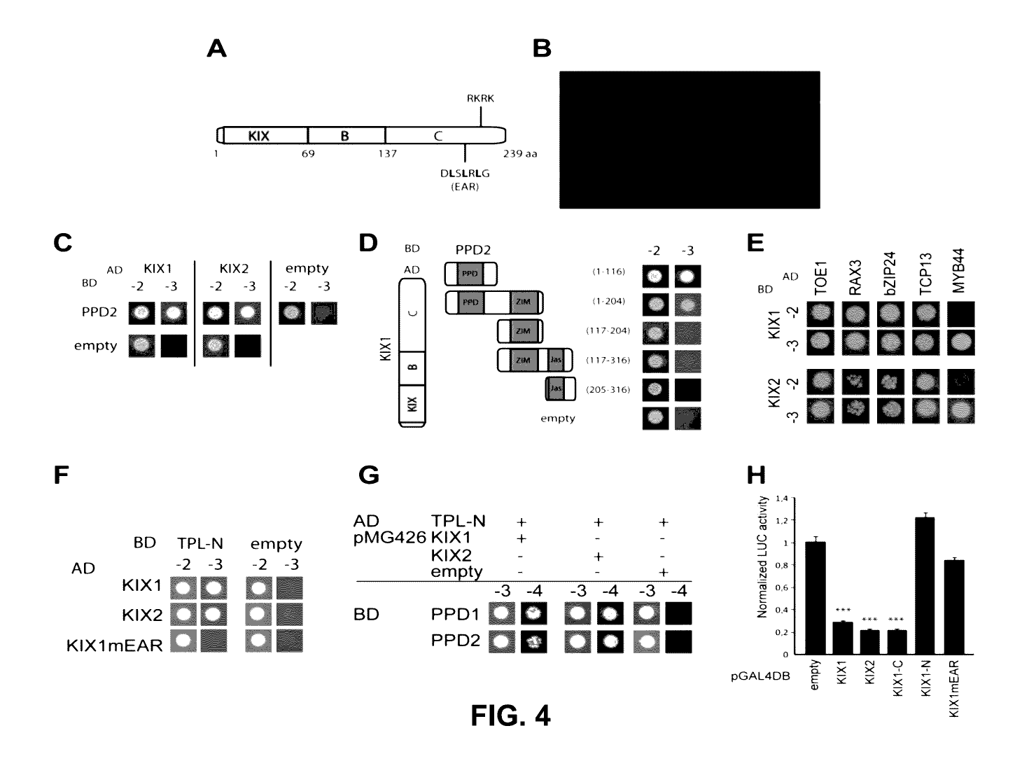

PPD2 interacting proteins, KIX8 and KIX9, are uncharacterized proteins that belong to the KIX protein family composed of ten members in Arabidopsis (Thakur et al., 2013). In fungi and metazoans, the KIX domain, mediating protein-protein interactions, is found in the multi-domain transcriptional activator histone acetyl transferase p300/CBP and in MED15 (Mediator subunit) where it interacts with several transcription factors (ref). The KIX proteins are, therefore, co-factors for transcriptional activation. KIX8 and KIX 9 were described to contain a KIX domain in their N-terminal region. By using Plaza (Van Bel et al., 2011), the different KIX protein orthologues were aligned from eudicots and could identify three extra regions (see FIG. 4, Panel A): a conserved domain B (aa 70-137 in KIX9) next to the N-terminus and highly conserved KIX domain (1-69), and in C-terminus, less conserved, an ERF-associated amphiphilic repression (EAR) motif (212-220) and a putative nuclear localization signal (NLS, 228-231). Nuclear localization could indeed be confirmed in Arabidopsis seedlings expressing KIX8-GFP (see FIG. 4, Panel B). Fluorescence was seen only in the nucleus, corresponding with the observed nuclear localization of PPD2 (Lacatus and Sunter 2009). To get more insight in the KIX-related protein complex, a TAP experiment was performed with KIX8, which confirmed the interaction in vivo with PPD2 (see Table 3). Y2H assays confirmed the direct interaction between the PPD and the KIX proteins (see FIG. 4, Panel C) and identified the N-terminal PPD domain of PPD2 as necessary and sufficient for the interaction with KIX9 (see FIG. 4, Panel D).

Interestingly, TAP of KIX8 revealed interaction with the RNA polymerase subunit B2 (NRPB2) (see Table3). Importantly, the NRPB2 orthologue in human has recently been shown to interact with the KIX-domain of RecQL5 (Islam et al., 2012).

Finally, an interaction was found with the uncharacterized TF SCARECROW-like 5, which also contains an EAR domain (Kagale et al., 2010). Interestingly, other TFs (TOE1, RAX3, BZIP24, TCP13) were reported to interact with KIX9 (The Arabidopsis Interactome Mapping Consortium (2011)) of which TOE1 also has an EAR motif (Kagale et al., 2010). Interaction of both KIX9 and KIX8 was confirmed with TOE1, RAX3, BZIP24, TCP13 but not with the random TF MYB44 (see FIG. 4, Panel E). The EAR motif, present in KIX proteins (see FIG. 4, Panel A), is known to mediate binding with the co-repressor TOPLESS. To confirm the direct interaction between the KIX proteins and TPL, Y2H assays were performed. KIX8 and KIX9 interacted directly with the LisH-domain of TPL and the C-terminus of KIX proteins was both necessary and sufficient (see FIG. 4, Panel F). Mutation of the Leu-residues in the LxLxL core to Ala abolished TPL interaction (see FIG. 4, Panel F).

To test whether the KIX proteins might have repressor activity, either of them was fused to the GAL4 DNA binding domain (GAL4DBD) and co-expressed with a construct expressing the firefly luciferase (fLUC) reporter gene under the control of GAL4 binding elements in tobacco bright-yellow (BY2) protoplasts. Both KIXs were capable of strong repression mediated by the EAR motif (see FIG. 4, Panel G). Finally, testing was performed to determine if KIX was capable of forming molecular bridge between PPD2 and TPL. Due to the absence of an EAR motif within its sequence, PPD2 is unable to bind directly to TPL (see FIG. 4, Panel H). Co-expression with either KIX8 or KIX9 is sufficient for GAL4 reconstitution, providing evidence for PPD2/KIX/TPL ternary complexes (see FIG. 4, Panel H). In conclusion, it was shown that PPD2 interacts with several protein partners and identified direct interacting proteins such as KIX8 and KIX9.

### 7. Double Kix8-Kix 9 Mutant Phenocopy Ami-Ppd Leaf Phenotype

In order to study the potential role of the KIX proteins on leaf growth, single T-DNA insertion lines for KIX8 (GABI_422H04) and KIX9 (SAIL_1168_G09) were obtained and kix8-kix9 double homozygous lines were generated. Plants were grown for 25 DAS in soil and leaf series done on wild-type, ami-ppd, kix8, kix9 and kix8-9 lines.

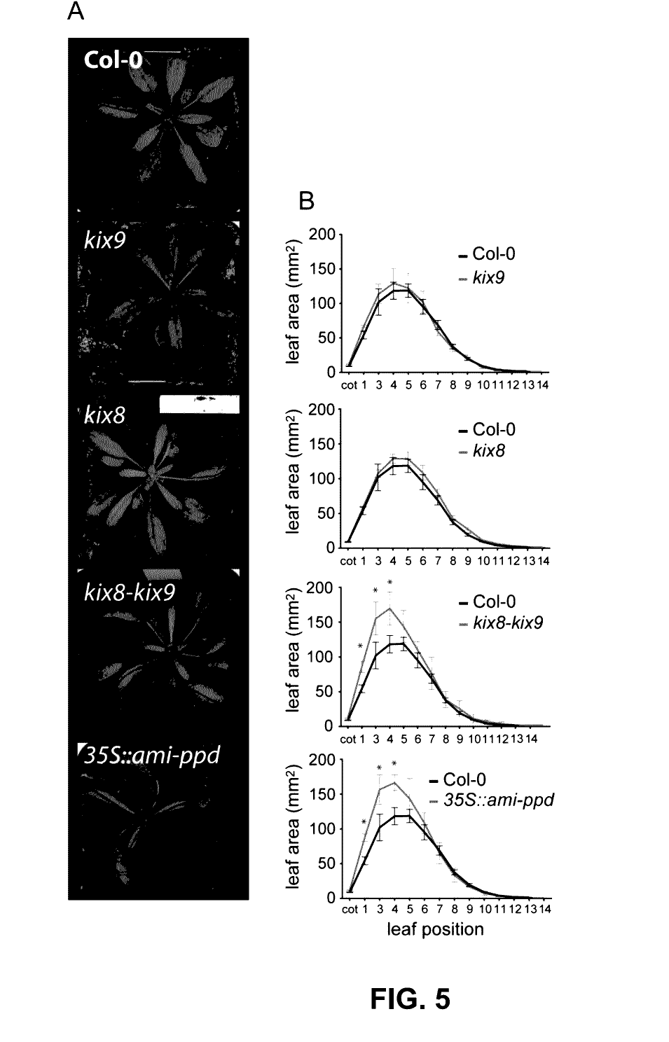

As shown in FIG. 5, Panel A, at rosette level, the dome-shaped phenotype previously described for the leaves of the ami-ppd line is also observed in the kix8-kix9 double mutant and to a lesser extent in the kix9 single mutant but not in the kix8 T-DNA insertion line. From the leaves series analysis, a similar increase in leaf area was found for the ami-ppd line and the kix8-kix9 double mutant whereas no increase is observed when only one of the two KIX is down-regulated. In conclusion, this analysis shows that the double kix8-kix9 mutant phenocopies the phenotype of the ami-ppd line, whereas the single lines do not have changes in leaf size.

8. PPD2 Target Genes are Mis-Expressed in Kix8-Kix9 Mutants and Require the Presence of KIX9 is Required for the Regulation of their Expression by PPD2

In order to estimate the involvement of the KIX proteins in the regulation of the expression of PPD2 targets, wild-type, single kix8, kix9 and double kix8-kix9 mutants were grown in vitro and leaves 1-2 were harvested at 11, 13 and 15 days after stratification for RNA extraction. mRNA levels of several PPD2 target genes (DFL1, SMZ, CYCD3;2, CYCD3;3 and HMGA) were quantified by qRT-PCR.

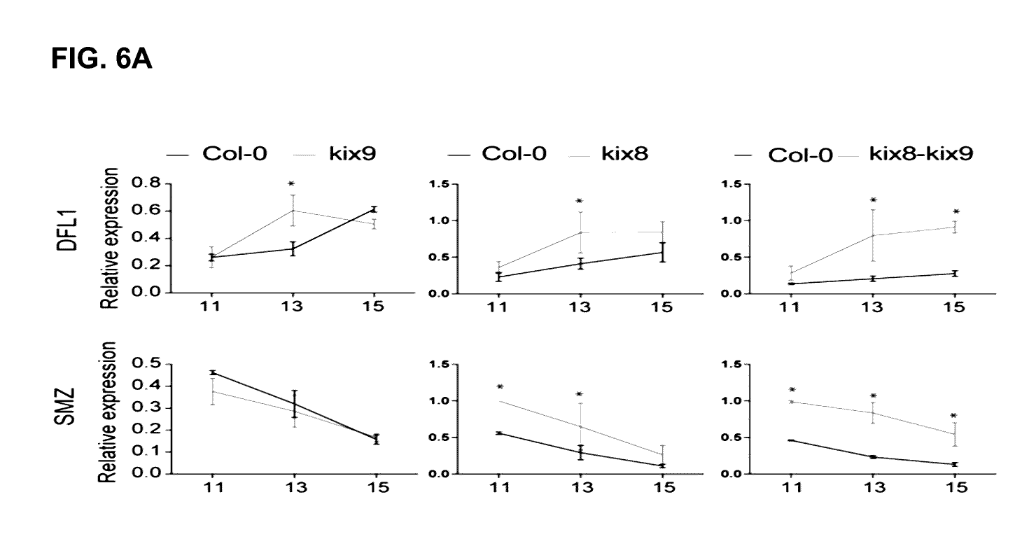

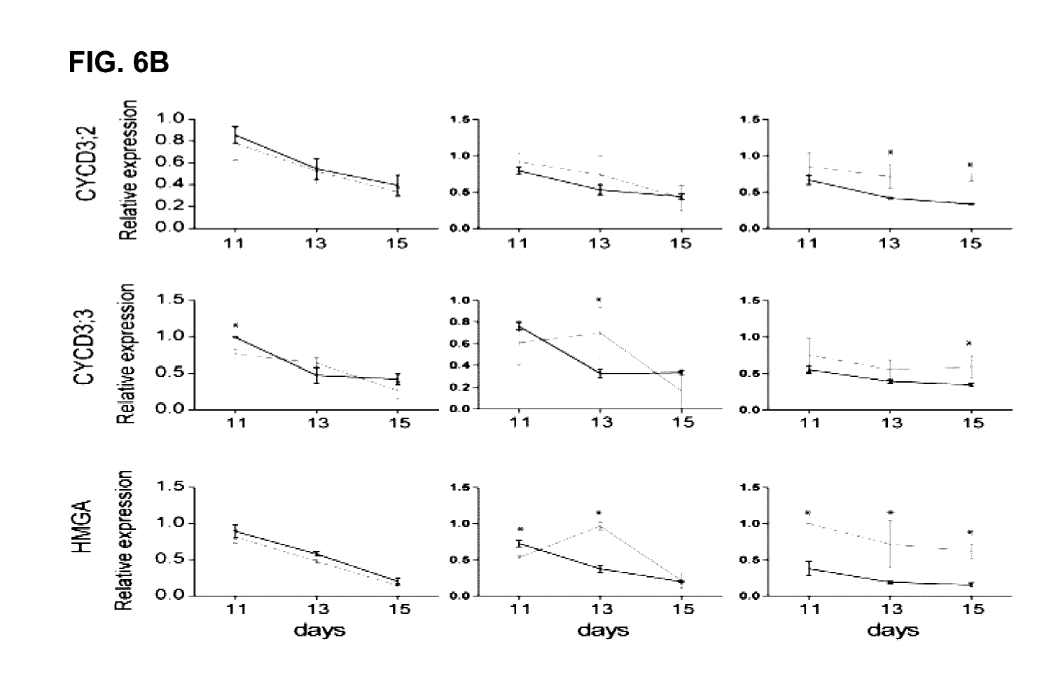

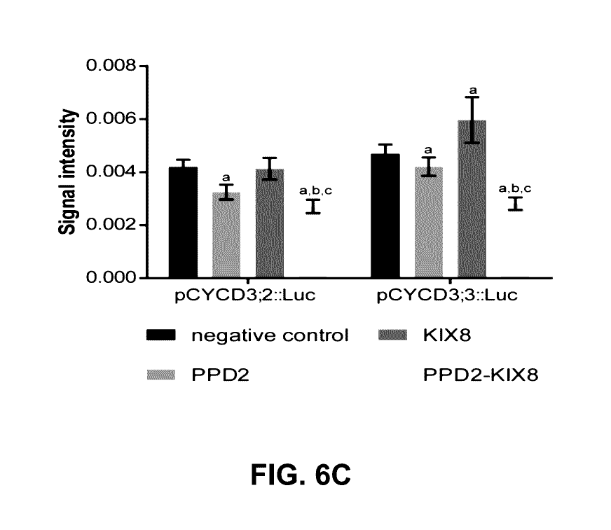

In kix9 leaves, none of the gene tested showed an obvious change in expression compared to wild-type except DFL1 having higher expression levels at 13 DAS (FIGS. 6A and 6B). In kix8 leaves, the expression of all tested genes, except CYCD3;2, is significantly increased compared to wild-type. As found in the ami-ppd line, the expression of DFL1, SMZ, CYCD3;2, CYCD3;3 and HMGA is significantly increased in the double kix8, kix9 mutant. In the kix8 mutant, leaves show a dome-shaped phenotype, such as ami-ppd, and the expression of the target genes of PPD2 is affected. Therefore, in order to analyze the contribution of KIX8 in the function of PPD2, a protoplast activation assay was performed with promoter-luciferase reporter constructs, in which promoters of PPD2 target genes are cloned upstream of the fLUC gene (encoding the firefly luciferase enzyme, pCYCD3;2:LUC and pCYCD3;3:LUC) and were expressed together with a 35S::PPD2 construct alone or in the presence of a 35S::KIX9 construct in tobacco (Nicotiana tabacum) Bright Yellow-2 protoplasts. Binding of PPD2 to the promoter of interest should trigger or inhibit the expression of the LUC gene and the production of the luciferase enzyme, which can be quantified by measuring luminescence. It was found that when pCYCD3;2:LUC and pCYCD3;3:LUC constructs are co-transformed with 35S::PPD2 construct, a slight decrease of the Luciferase signal is observed, compared to control (23% and 10%, respectively) (FIG. 6C). When these constructs are co-transformed with the 35S::KIX8 vector, no change or a slight increase of the luciferase signal is observed in the combination pCYCD3;2:LUC-35S::KIX8 and pCYCD3;3:LUC-35S::KIX8, respectively. On the other hand, when the PPD2 and KIX2 are co-expressed in the protoplasts, the luciferase signal for pCYCD3;2:LUC and pCYCD3;3:LUC decreases compared to control (35% and 40%, respectively), 35S::PPD2 (17% and 34%, respectively) and 35S::KIX8 (35% and 53%, respectively).

Taken together, these data suggest that regulation of leaf growth by PPD2 and the expression of its target genes is dependent on the presence of the KIX proteins.

### 9. Identification of Plant Orthologous Genes of KIX8

Glycine max:
GM13G22660 (53.6% similarity, 42.3% identity) (SEQ ID NO:5)
Other orthologous genes present in Glycine max:
GM 17g12140.2 (47.5% similarity, 39.1% identity)
GM 04g07060.1 (45.8% similarity, 35% identity)

Gossypium raimondii:
GR010G062400.1 (56.6% similarity, 44.9% identity) (SEQ ID NO:6)
Other orthologous genes:
GR009G058400.1 (51.9% similarity, 41.1% identity)
GR001G033800.1 (51.6% similarity, 38.2% identity)

Populus trichocarpa:
PT006G067100.1 (49% similarity, 39.1% identity) (SEQ ID NO. 7)
Other orthologous genes:
PT018G128700.1 (48.4% similarity, 37.3% identity)
PT006G254700.1 (44% similarity, 36.4% identity)

### 10. Identification of Orthologous Genes of KIX9

Glycine max:
GM04g07060.1 (57.1% similarity, 46.2% identity) (SEQ ID NO:8)
Other orthologous genes:
GM06g07151.1 (56.3% similarity, 46.6% identity)
GM13g22660.1 (52.9% similarity, 44.5% identity)
GM06g26601.1 (63% similarity, 50% identity)

Gossypium raimondii (10):
GR009G058400.1 (51.3% similarity, 47.1% identity) (SEQ ID NO. 9)
Other orthologous genes:
GR001G033800.1_(52.9% similarity, 45% identity)

Populus trichocarpa (5):
PT006G254700.1 (54.6% similarity, 49.6% identity), PT.006G254700.2 (SEQ ID NO:10)
Other orthologous genes:
PT018G027100.1 (53.8% similarity, 49.2% identity)
PT018G128700.1 (55% similarity, 43.3% identity)

### REFERENCES

(1) Jan, Y. N., and L. Y. Jan (1998). Asymmetric cell division. Nature 392:775-778.
(2) Scheres, B., and P. N. Benfey (1999). Asymmetric cell division in plants. Annu. Rev. Plant Physiol. Plant Mol. Biol. 50:505-537.
(3) Ohashi-Ito, K., and D. C. Bergmann (2006). Arabidopsis FAMA controls the final proliferation/differentiation switch during stomatal development. Plant Cell 18:2493-2505.
(4) MacAlister, C. A. et al. (2007). Transcription factor control of asymmetric cell divisions that establish the stomatal lineage. Nature 445:537-540.
(5) Pillitteri, L. J. et al. (2007). Termination of asymmetric cell division and differentiation of stomata. Nature 445:501-505.
(6) Nadeau, J. A., and F. D. Sack (2002). Stomatal development in Arabidopsis. In The Arabidopsis Book (eds. C. Somerville and E. Meyerowitz) doi:10.1199/tab.0066 (American Society of Plant Biologists, 2002).
(7) Geisler, M. et al. (2000). Oriented asymmetric divisions that generate the stomatal spacing pattern in Arabidopsis are disrupted by the too many mouths mutation. Plant Cell 12:2075-2086.
(8) Skirycz, A. et al. (2011). Pause-and-stop: The effects of osmotic stress on cell proliferation during early leaf development in Arabidopsis and a role for ethylene signaling in cell cycle arrest. Plant Cell 23:1876-1888.
(9) Bergmann, D. C. et al. (2004). Stomatal development and pattern controlled by a MAPKK kinase. Science 304:1494-97.
(10) White, D. W. R. (2006). PEAPOD regulates lamina size and curvature in Arabidopsis. Proc. Natl. Acad. Sci. U.S.A. 103:13238-13243.
(11) Zhang, Y. et al. (2012). Genome-wide identification and analysis of the TIFY gene family in grape. PLoS One 7(9):e44465. doi: 10.1371
(12) Pauwels, L. et al. (2010). NINJA connects the co-repressor TOPLESS to jasmonate signaling. Nature 464:788-791.

1. A dicotyledonous plant, wherein the expression of the KIX8 gene and the expression of the KIX9 gene have been reduced by at least 80%.

2. The dicotyledonous plant according to claim 1 having a loss of function of the KIX8 and KIX9 genes.

3. The dicotyledonous plant according to claim 1, which has a gene disruption in genes KIX8 and KIX9.

4. A seed or a plant cell derived from the plant according to claim 1.

5. A method of producing a dicotyledonous plant having at least a 30% reduction in the expression of the KIX8 and KIX9 genes, the method comprising:
introducing into the dicotyledonous plant a silencing RNA construct directed to genes KIX8 and KIX9 or an artificial microRNA directed to genes KIX8 and KIX9.

6. A method of increasing the yield of a dicotyledonous plant by at least 50%, the method comprising:
introducing a silencing RNA construct directed to genes KIX8 and KIX9 or an artificial microRNA directed to genes KIX8 and KIX9 into the dicotyledonous plant.

7. A recombinant vector comprising:
a silencing RNA construct directed to genes KIX8 and KIX9; or
an artificial microRNA directed to genes KIX8 and KIX9.

8. A plant or plant cell or plant seed comprising a recombinant vector according to claim 7.

9. The dicotyledonous plant of claim 2, wherein genes KIX8 and KIX9 have been disrupted in the plant.

10. A seed or a plant cell derived from the plant of claim 2.

11. A seed or a plant cell derived from the plant of claim 3.

12. A seed or a plant cell derived from the plant of claim 9.
